(19) United States
(12) Patent Application Publication
(10) Pub. No.: US 2017/0000596 A1
(43) Pub. Date: Jan. 5, 2017

(54) PUNCTURE MEMBER

(71) Applicant: TERUMO KABUSHIKI KAISHA, Tokyo (JP)

(72) Inventors: Yuusuke TAKAHASHI, Hadano-shi (JP); Masakatsu KAWAURA, Sunnyvale, CA (US); Nao MANDAI, Mountain View, CA (US)

(73) Assignee: TERUMO KABUSHIKI KAISHA, Tokyo (JP)

(21) Appl. No.: 15/264,269

(22) Filed: Sep. 13, 2016

Related U.S. Application Data
(63) Continuation of application No. PCT/JP2015/055353, filed on Feb. 25, 2015.

(30) Foreign Application Priority Data
Mar. 13, 2014 (JP) 2014-050884

(51) Int. Cl. A61F 2/00; A61B 17/34

(52) U.S. Cl. CPC A61F 2/0045; A61B 17/3468; A61B 17/3403; A61B 2017/00805

(57) ABSTRACT

A puncture member is disclosed, which includes a needle body which punctures a living body; a shaft portion which is located on a proximal side of the needle body and has a first shaft portion to be inserted into the living body together with the needle body; and an interlock portion which is provided at a distal portion of the first shaft portion and interlocks the first shaft portion and the needle body in a freely detachable manner.

### CROSS-REFERENCES TO RELATED APPLICATIONS

This application is a continuation of International Application No. PCT/JP2015/055353, filed on Feb. 25, 2015, which claims priority to JP 2014-050884, filed on Mar. 13, 2014, the entire content of which is incorporated herein by reference.

### TECHNICAL FIELD

The present disclosure relates to a puncture member.

### BACKGROUND DISCUSSION

In a patient suffering from urinary incontinence, particularly stress urinary incontinence, urine leakage can occur due to an abdominal pressure exerted during a normal exercise, by laughing, coughing, or sneezing. This can be attributable, for example, to loosening of the pelvic floor muscle, which is a muscle for supporting the urethra, caused by childbirth or the like.

For the treatment of urinary incontinence, surgical therapy can be effective, in which there is used, for example, a belt-shaped living body tissue-supporting indwelling article called “sling.” The sling is placed indwelling in the body and the urethra is supported by the sling (see, for example, Japanese Patent Laid-open No. 2010-99499). In order to put a sling indwelling inside the body, an operator would incise the vagina with a surgical knife, dissect the living body tissue between the urethra and vagina, and make the dissected region and the outside communicate with each other through obturator foramens by use of a puncture needle. Then, in this state, the sling is placed indwelling in the body.

If the vagina is incised once, however, a situation may occur that the sling is exposed to the inside of the vagina from a wound caused by the incision of the vagina, and complications may be caused by an infection from the wound. Further, since the vagina is incised, there is such a defect that the invasion can be relatively great and the burden on the patient can be relatively heavy. Further, the urethra may be damaged in the course of the procedure by the operator. In addition, the fingertip of the operator may be damaged or injured.

### SUMMARY

Thus, there is a need for a puncture member with which a procedure of embedding a living body tissue-supporting indwelling article in a living body can be performed relatively safely.

A puncture member is disclosed, which includes: a needle body which punctures a living body; a shaft portion which is located on a proximal side of the needle body and has a first shaft portion to be inserted into the living body together with the needle body; and an interlock portion which is provided at a distal portion of the first shaft portion and interlocks the first shaft portion and the needle body in a freely detachable manner.

The puncture member may have a configuration wherein when the first shaft portion is moved proximally in a condition where the needle body is located in the living body, the needle body is moved proximally together with the first shaft portion while retaining a state of being interlocked with the first shaft portion.

Preferably, the puncture member further includes a restriction unit which restricts proximal movement of the needle body while permitting proximal movement of the first shaft portion, and the needle body and the first shaft portion are detached from each other when the first shaft portion is moved proximally in a condition where proximal movement of the needle body is restricted by the restriction unit.

In the puncture member, preferably, one of the needle body and the interlock portion is provided with a projection, whereas the other of the needle body and the interlock portion is provided with an interlock hole for interlocking with the projection, and the first shaft portion and the needle body are interlocked to each other by insertion of the projection into the interlock hole.

The puncture member may have a configuration wherein: the projection includes an elastically deformable portion which can be contracted in width as compared to a natural state by elastic deformation; the interlock hole includes an introduction portion having a width smaller than the width of the elastically deformable portion in the natural state, and an enlarged width portion which is located on a depth side of the introduction portion and is greater in width than the introduction portion; and the first shaft portion and the needle body are interlocked to each other, by the elastically deformable portion being located in the enlarged width portion.

In the puncture member, preferably, the interlock portion includes a flat-shaped portion.

The puncture member may have a configuration wherein the shaft portion includes the first shaft portion and a second shaft portion slidable relative to the first shaft portion, and the shaft portion is extendable by sliding of the first shaft portion and the second shaft portion.

Preferably, the puncture member further includes a pipe-shaped medical tube in which the shaft portion is inserted, and the needle body is connected to a distal portion of the medical tube.

The puncture member may have a configuration wherein: the medical tube includes an outer tube, and an inner tube which is disposed inside the outer tube and is slidable relative to the outer tube, the medical tube being extendable by sliding of the outer tube and the inner tube; and the needle body is connected to a distal portion of the inner tube.

In another aspect, a puncture apparatus is disclosed, which includes: a needle body; a shaft portion including a first shaft portion which is located on a proximal side of the needle body and is interlocked to the needle body in a freely detachable manner and a second shaft portion interlocked to the first shaft portion in a slidable manner; a restriction unit establishing a restriction state in which proximal movement of the second shaft portion is restricted while proximal movement of the first shaft portion is permitted; and a support portion which supports the shaft portion.

In the puncture apparatus, preferably, the restriction unit is movable relative to the support portion in an initial state, and comes into the restriction state by being fixed to the support portion.

In the puncture apparatus, preferably, the restriction unit is fixed to the support portion when the first shaft portion and the second shaft portion are both moved distally starting from the initial state.

In the puncture apparatus, preferably, the restriction unit is interlocked to the first shaft portion in a slidable manner.

Preferably, the puncture apparatus further includes an interlock portion which is interlocked to a proximal portion of the first shaft portion and restricts detachment of the restriction unit from a proximal side of the first shaft portion, and the restriction unit is moved distally together with the first shaft portion by being pushed by the interlock portion.

Preferably, the puncture apparatus includes a male-type engaging portion provided on one of the restriction unit and the support portion, and a female-type engaging portion provided in the other of the restriction unit and the support portion, and the restriction unit is fixed to the support portion by engagement between the male-type engaging portion and the female-type engaging portion.

The puncture apparatus may further include a pipe-shaped medical tube into which the shaft portion is inserted.

In the puncture apparatus, preferably, the restriction unit is located on a proximal side of the medical tube, and makes contact with a proximal portion of the medical tube in the restriction state.

In the puncture apparatus, preferably, the medical tube includes an outer tube, and an inner tube located inside the outer tube and slidable relative to the outer tube, the medical tube being extendable by sliding of the outer tube and the inner tube.

In a further aspect, there is disclosed a puncture member including: a needle body; a shaft portion including a first shaft portion which is located on a proximal side of the needle body and is interlocked to the needle body in a freely detachable manner, and a second shaft portion provided in alignment with the first shaft portion; and an interlock portion which is provided at a distal portion of the first shaft portion, retains the second shaft portion in a slidable manner, and restricts relative positional relation between the first shaft portion and the second shaft portion.

In the puncture member, preferably, the interlock portion maintains a separated distance between the first shaft portion and the second shaft portion within a predetermined distance in a vicinity of the interlock portion.

The puncture member may have a configuration wherein the interlock portion has at least a pair of insertion holes spaced apart along an extending direction of the second shaft portion, the second shaft portion is inserted in and passed through the pair of insertion holes, and the separated distance between the first shaft portion and the second shaft portion is maintained within the predetermined distance between the pair of insertion holes.

In the puncture member, preferably, the interlock portion restricts distal movement of the first shaft portion relative to the second shaft portion after an extended state in which the shaft portion is extended is established by proximal movement of the first shaft portion relative to the second shaft portion starting from an initial state.

The puncture member may have a configuration wherein the interlock portion permits proximal movement of the first shaft portion relative to the second shaft portion after the extended state is established, and the interlock portion is detached from the second shaft portion by proximal movement of the first shaft portion relative to the second shaft portion.

Preferably, the puncture member further includes a contact surface which is provided on one of the interlock portion and the second shaft portion and is oriented toward a proximal side of the second shaft portion, and a contact portion which is provided on the other of the interlock portion and the second shaft portion and makes contact with the contact surface from the proximal side, and the contact surface and the contact portion contact or face each other in the extended state.

In the puncture member, preferably, the interlock portion is provided with the contact portion, and the second shaft portion is provided with the contact surface.

In the puncture member, preferably, the contact portion is located between the pair of insertion holes.

In the puncture member, preferably, the interlock portion includes a portion having a flat shape.

Preferably, the puncture member has a pipe-shaped medical tube into which the shaft portion is inserted, the medical tube has a flat cross-sectional shape having a minor axis and a major axis, and the first shaft portion and the second shaft portion are aligned within the medical tube along the major-axis direction of the medical tube.

In the puncture member, preferably, the needle body is connected to a distal portion of the medical tube.

In yet another aspect, there is disclosed a puncture member including: a pipe-shaped medical tube; and a needle body connected to a distal portion of the medical tube, wherein the needle body has an enlarged width portion, and the width of the enlarged width portion is greater than the width of the distal portion of the medical tube.

In the puncture member, preferably, the needle body has, on a proximal side of the enlarged width portion, a contact surface for making contact with the distal portion of the medical tube.

The puncture member may have a configuration wherein the medical tube includes an outer tube, and an inner tube disposed inside the outer tube and slidable relative to the outer tube, and at least one of the outer tube and the inner tube is provided with the contact surface.

In the puncture member, preferably, the medical tube is extendable by sliding of the outer tube and the inner tube.

In the puncture member, preferably, a distal portion of the outer tube is inclined toward inside of the medical tube.

In the puncture member, preferably, the width of the enlarged width portion is greater than the width of the distal portion of the outer tube, and the width of the distal portion of the outer tube is greater than the width of a distal portion of the inner tube.

The puncture member may have a configuration wherein the needle body includes a needle portion having the enlarged width portion, and a proximal portion located on a proximal side of the needle portion, and the proximal portion is inserted in the medical tube.

In the puncture member, preferably, the enlarged width portion is capable of dissecting a living body tissue.

In the puncture member, preferably, the needle body is provided, between a distal end thereof and a proximal end thereof, with the enlarged width portion which is greater in width than on a distal side thereof and than on a proximal side thereof.

In the puncture member, preferably, the needle body includes a gradually increasing width portion which is located on a proximal side of the enlarged width portion and the width of which gradually increases toward a distal side.

In the puncture member, preferably, the needle body has a flat cross-sectional shape having a minor axis and a major axis.

In the puncture member, preferably, the cross-sectional shape of the needle body is a fusiform wherein a central portion in regard of the major axis is swollen.

Preferably, the puncture member includes an elongate shaft portion inserted in the medical tube, and the shaft portion and the needle body are connected to each other in a freely detachable manner.

According to the present disclosure, the needle body and the first shaft portion are freely detachable from each other. Therefore, the puncture member having punctured a living body can be removed relatively easily. In the case where the shaft portion has the first shaft portion and the second shaft portion and is extendable, the extension of the shaft portion can be reliably performed. In the case where the first shaft portion can be moved proximally while retaining the state of being interlocked with the needle body, the needle body is not detached, even if the puncture member is retracted for correction of a puncture route of the puncture member, after the puncture route is positionally deviated, for example. Therefore, the retraction of the puncture member can be carried out relatively easily, and the needle body can be prevented from being left inside a living body. Accordingly, a procedure of embedding a living body tissue-supporting indwelling article into a living body can be performed relatively smoothly and safely.

### BRIEF DESCRIPTION OF THE DRAWINGS

FIG. 1 is a perspective view of an example of an implant;

FIG. 2 is a perspective view of a puncture apparatus according to an exemplary embodiment;

FIG. 3 is a side view of the puncture apparatus shown in FIG. 2;

FIG. 4 is a plan view (sectional view) of a puncture member possessed by the puncture apparatus shown in FIG. 2;

FIG. 5 is a partial enlarged view of FIG. 4;

FIGS. 6A and 6B illustrate a sheath possessed by the puncture member shown in FIG. 4, wherein FIG. 6A is a perspective view, and FIG. 6B is a sectional view taken along line VIB-VIB of FIG. 6A;

FIG. 7 is a sectional view taken along line VII-VII of FIG. 4;

FIG. 8 is a plan view (sectional view) for illustrating an operation of the puncture member shown in FIG. 4;

FIG. 9 is a plan view (sectional view) for illustrating an operation of the puncture member shown in FIG. 4;

FIG. 10 is a plan view (sectional view) for illustrating an operation of the puncture member shown in FIG. 4;

FIG. 11 is a plan view of a fixation portion of a frame possessed by the puncture apparatus shown in FIG. 2;

FIG. 12 is a side view of an insertion device possessed by the puncture apparatus shown in FIG. 2;

FIGS. 13A and 13B illustrate a positional relation between the puncture member and obturator foramens (pelvis), wherein FIG. 13A is a side view, and FIG. 13B is a front view;

FIG. 14 is a top plan view of a distal portion possessed by a vaginal-insertion device shown in FIG. 12;

FIG. 15A is a sectional view of an example of the shape of a vaginal wall, and FIG. 15B is a sectional view showing a state wherein a vaginal-insertion portion is inserted in the vagina shown in FIG. 15A;

FIGS. 16A and 16B are views for illustrating an operation procedure of the puncture apparatus shown in FIG. 2;

FIG. 17 is a view for illustrating an operation procedure of the puncture apparatus shown in FIG. 2;

FIG. 18 is a view for illustrating an operation procedure of the puncture apparatus shown in FIG. 2;

FIG. 19 is a view for illustrating an operation procedure of the puncture apparatus shown in FIG. 2;

FIG. 20 is a view for illustrating an operation procedure of the puncture apparatus shown in FIG. 2;

FIG. 21 is a view for illustrating an operation procedure of the puncture apparatus shown in FIG. 2;

FIG. 22 is a view for illustrating an operation procedure of the puncture apparatus shown in FIG. 2;

FIGS. 23A and 23B are views for illustrating an operation procedure of the puncture apparatus shown in FIG. 2;

FIGS. 24A and 24B are views for illustrating an operation procedure of the puncture apparatus shown in FIG. 2; and

FIGS. 25A and 25B are views for illustrating an operation procedure of the puncture apparatus shown in FIG. 2.

### DETAILED DESCRIPTION

A puncture member according to the present disclosure will be described in detail below, on the basis of preferred embodiments shown in the accompanying drawings.

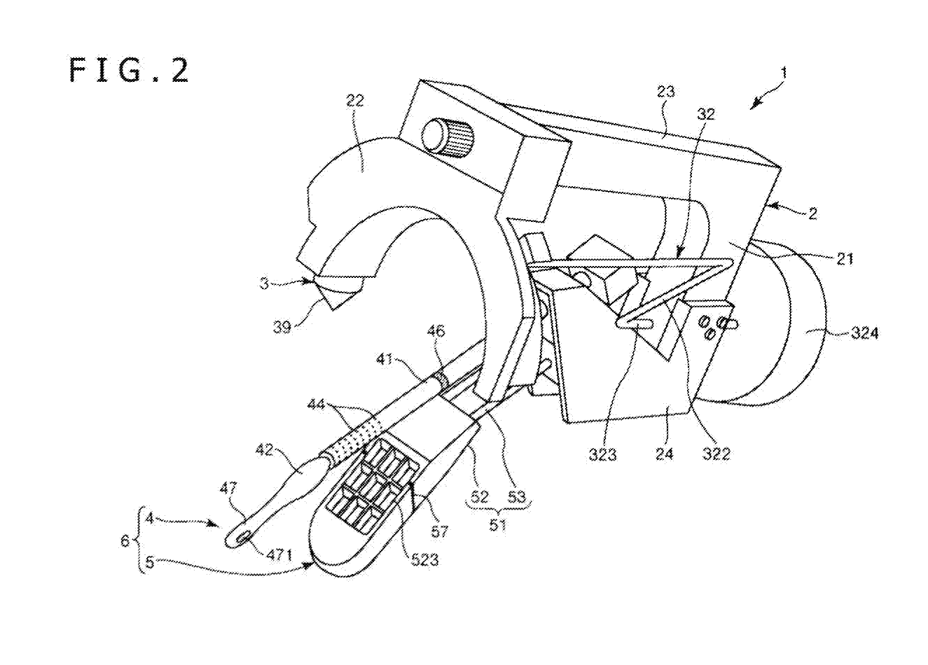

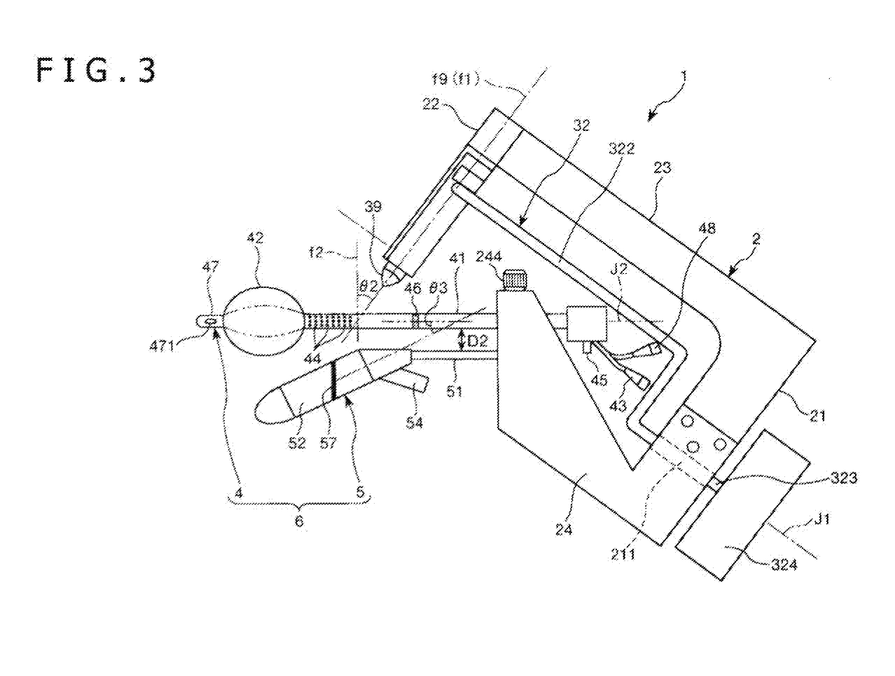

Note that in the following, for convenience of explanation, the left side in FIG. 3 will be referred to also as “distal end,” the right side in FIG. 3 will be referred to also as “proximal end,” the upper side in FIG. 3 will be referred to also as “upper side,” and the lower side in FIG. 3 will be referred to also as “lower side.” In addition, FIGS. 2 to 4 illustrate a state wherein a puncture apparatus is not yet used (a state when starting the procedure), and this state will hereinafter be referred to also as “initial state” for convenience of explanation. In addition, a state wherein a puncture apparatus shown in FIGS. 2 and 3 is mounted onto a patient will be referred to also as “mounted state” for convenience of explanation.

1. Implant

In accordance with an exemplary embodiment, one example of an implant (living body tissue-supporting indwelling article) 9 to be embedded in a living body by a puncture apparatus will be described.

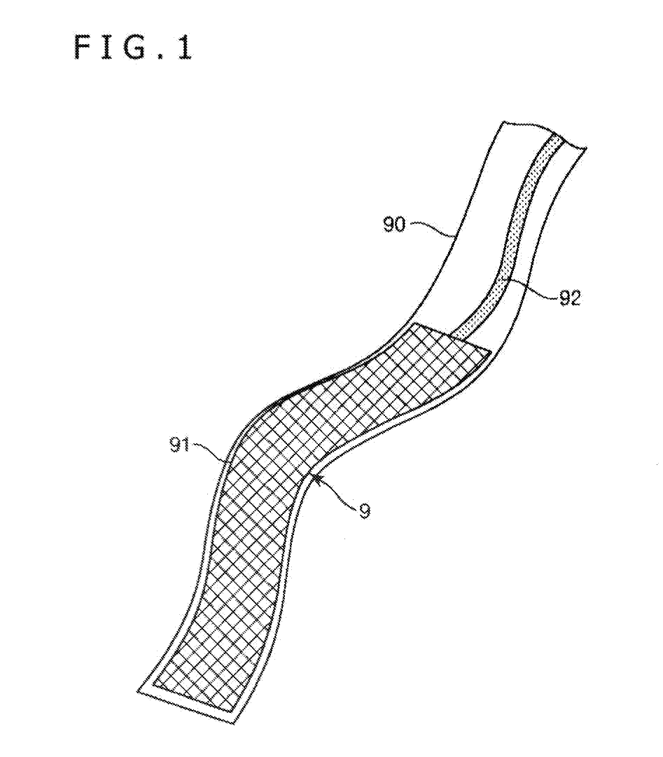

The implant 9 illustrated in FIG. 1 is a device which can be embedded in a living body for treatment of female urinary incontinence. More specifically, the implant 9 is a device for supporting a urethra, for example, a device which, when the urethra is going to move toward the vaginal-wall side, supports the urethra so as to restrict such a movement, in the manner of pulling the urethra in a direction for spacing away from the vaginal wall. As such an implant 9, for example, an elongate body having flexibility can be used.

As shown in FIG. 1, the implant 9 can include an implant main body 91, and a belt (connection portion) 92 connected to one end of the implant main body 91. Note that, for example, a guide wire, a cord, a string or the like may be used in place of the belt 92. The implant main body 91 is net-like in form, and is belt-like in overall shape. The implant main body 91 may be composed, for example, of a net-like knitted body formed by intersecting linear (filamentous) members, or a net-like braiding. Examples of the linear (filamentous) member include those which are circular in cross-sectional shape, and those which are flattened in cross-sectional shape, namely, tape-like members.

The implant 9 configured in this way can be accommodated in a sterilized wrapping material 90 in its unused state. By this, the implant 9 can be prevented against contamination.

Respective constituent materials of the implant main body 91, the belt 92 and the wrapping material 90 are not particularly limited. Examples of usable materials include various biocompatible resin materials such as polypropylene, polyester, nylon, and fibers.

While the implant 9 has been described above, the implant 9 is not limited to the net-like one, so long as the implant 9 can exhibit the equivalent or similar effects to the above-mentioned.

2. Puncture Apparatus

A puncture apparatus 1 illustrated in FIGS. 2 and 3 is an apparatus for use in embedding the aforementioned implant 9 into a living body.

As depicted in FIGS. 2 and 3, the puncture apparatus 1 can include a frame 2, an insertion device 6, and a puncture member 3, wherein the puncture member 3 and the insertion device 6 are supported by the frame 2. In addition, the insertion device 6 can include a urethral-insertion device 4 and a vaginal-insertion device 5.

Each of these components will be described below.

### Puncture Member

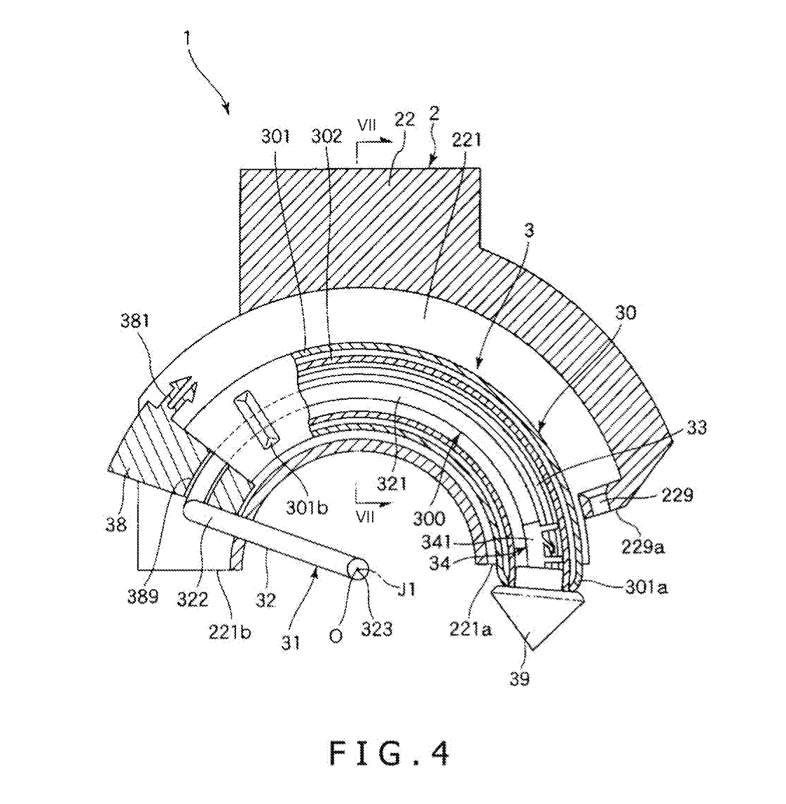

The puncture member 3 is a member for puncturing a living body. As shown in FIG. 4, the puncture member 3 can include an operation member 31, an elongate sheath (medical tube) 30 mounted to the operation member 31, a needle body 39 provided on a distal side of the sheath 30, and a slide lock unit (restriction unit) 38 provided on a proximal side of the sheath 30. The sheath 30, the needle body 39, the operation member 31 and the slide lock unit 38 will be sequentially described hereinbelow.

### Sheath

As illustrated in FIGS. 4 to 6B, the sheath 30 has an arcuate curved shape. In addition, the sheath 30 can be tubular in form, and the inside of the sheath 30 functions as a space in which to insert the operation member 31 (a stylet 321 and a pusher shaft 33 which will be described later) and as a space in which to insert the implant 9.

As depicted in FIG. 4, the sheath 30 can include an outer tube 301, and an inner tube 302 inserted in the outer tube 301, wherein the inner tube 302 is slidable relative to the outer tube 301. Therefore, the inner tube 302 can protrude through a distal side opening of the outer tube 301, from an initial state in which the inner tube 302 is retracted into the outer tube 301. As a result, an extendable sheath 30 is obtained. In addition, the inner tube 302 can be detached from the distal side opening of the outer tube 301, which can help ensure that the sheath 30 can be divided into the outer tube 301 and the inner tube 302, and the sheath 30 disposed in a living body can be relatively easily removed from the living body. Note that an outer surface of the inner tube 302 may be subjected to a friction-lowering treatment in order to enhance slidability of the inner tube 302 relative to the outer tube 301. In addition, the inner tube 302 may not necessarily be slidable toward the proximal side, so long as the inner tube 302 is slidable toward the distal side of the outer tube 301. For example, the sheath 30 may not necessarily be contractible from an extended state, so long as the sheath 30 can be extended from the initial state.

In accordance with an exemplary embodiment, as shown in FIG. 4, the outer tube 301 is provided at a proximal portion of the outer tube 301 with a contact portion 301b for making contact with a body surface H. The contact portion 301b is composed of a projection piece provided to project from the outer tube 301. When the puncture member 3 (sheath 30) punctures a living body, the contact portion 301b makes contact with the body surface H, to function as a stopper which inhibits the outer tube 301 from being inserted further into the living body. By this, the proximal side opening of the outer tube 301 can be prevented from being embedded in the living body and thereby hidden.

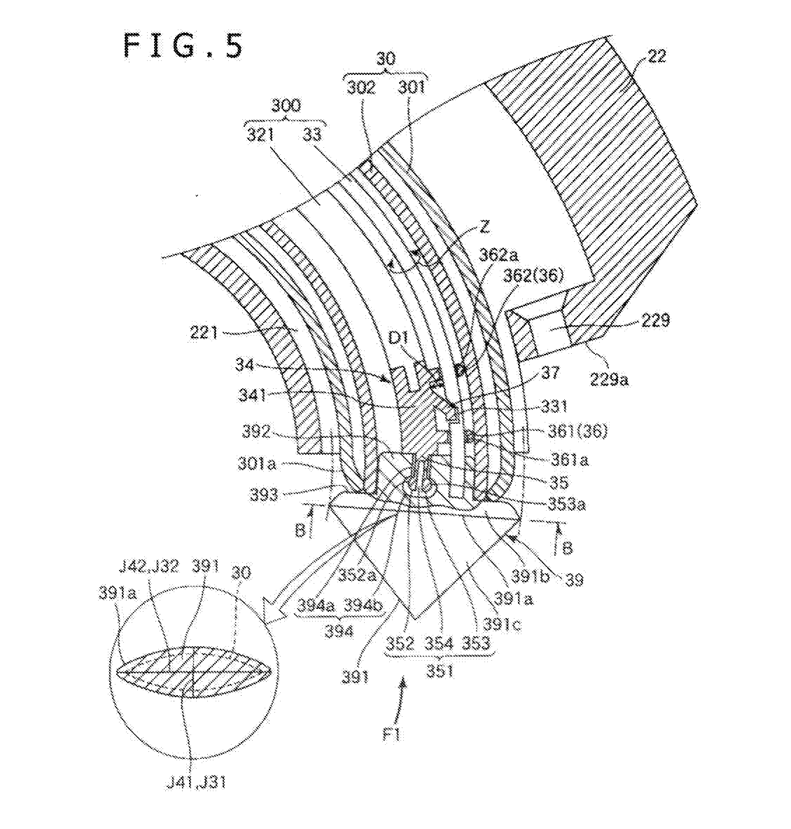

In addition, as shown in FIG. 5, a distal portion 301a of the outer tube 301 constitutes an inclined surface which is inclined toward the inner side (center axis side), and has a tapered shape contracted in diameter (contracted in width) along a distal direction. The distal portion 301a of the outer tube 301 is in secure contact with an outer circumference of the inner tube 302 such that no substantial gap is formed between the distal portion 301a and the outer circumference of the inner tube 302, which helps ensure that when the puncture member 3 punctures a living body, living body tissue does not easily penetrate between the outer tube 301 and the inner tube 302. Accordingly, a lowering in slidability of the inner tube 302 relative to the outer tube 301 can be restrained, and the inner tube 302 can be slid smoothly relative to the outer tube 301.

In addition, an effect as follows is also obtained. When the puncture member 3 punctures a living body, a stress F1 in the direction of an arrow mark in FIG. 5 is exerted on the needle body 39, and, depending on the magnitude of the stress F1 and the hardness of the outer tube 301, the distal portion 301a of the outer tube 301 may be deformed. Where the distal portion 301a of the outer tube 301 is preliminarily inclined to the inner side as in this embodiment, the distal portion 301a is deformed in the manner of being bent further to the inner side upon receiving the stress F1. Where the distal portion 301a is straight in shape, for example, the distal portion 301a may be deformed in the manner of being bent to the inner side, but may also be deformed in the manner of increasing in diameter to the outer side, upon receiving the stress F1. If the distal portion 301a is deformed to the outer side, the distal portion 301a of the outer tube 301 would protrude to the outside with respect to the needle body 39, and the protruding portion may be caught on the living body tissue. If the protruding portion is caught on the living body tissue, puncturing resistance on the puncture member 3 may be increased, whereby smooth puncturing may be hampered. Therefore, it can be desirable to form the distal portion 301a in a tapered shape as in this embodiment.

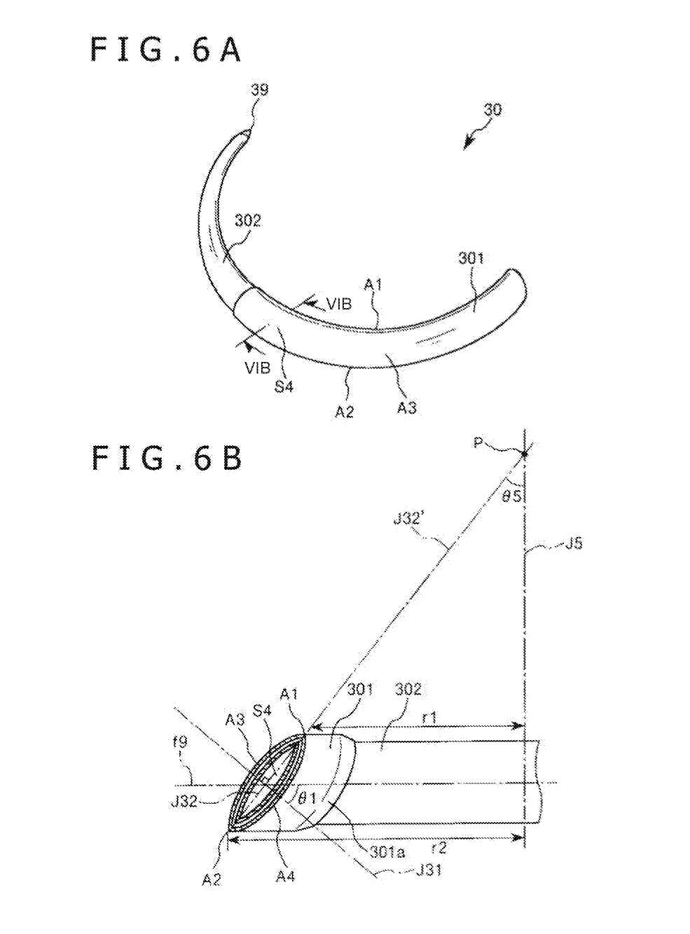

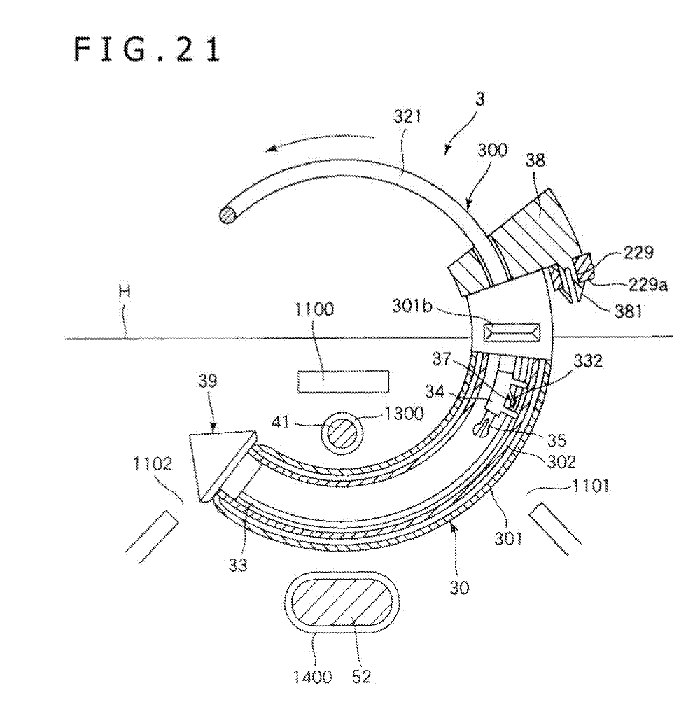

A center angle of the sheath 30 configured as above is not particularly limited, and is appropriately set according to various conditions. The center angle is set in such a manner that the needle body 39 can enter a living body via an inguinal region on one side of the patient, sequentially passes an obturator foramen on the one side, between the urethra and the vagina, and an obturator foramen on the other side, to protrude to the exterior of the living body via an inguinal region on the other side. Specifically, in the extended state in which the inner tube 302 is protruded from the outer tube 301 as shown in FIG. 6A and FIG. 21, the center angle, for example, is preferably approximately 150 degrees to 270 degrees, more preferably approximately 170 degrees to 250 degrees, and further preferably approximately 190 degrees to 230 degrees.

In addition, as illustrated in FIG. 6B, the sheath 30 has a flat cross-sectional shape having a minor axis J31 and a major axis J32. With the sheath 30 thus set to be flat in cross-sectional shape, the width direction of the implant main body 91 within the sheath 30 can be conformed to the width direction of the sheath 30, whereby the implant main body 91 can be controlled into a desired posture. Note that the flat shape of the sheath 30 is not specifically restricted. Examples of the applicable flat shape can include an ellipse, a convex lens-like shape in section, a rounded-cornered rhombus, a rounded-cornered rectangle (flat shape), and a fusiform with a central portion enlarged (radially enlarged) as compared with both end portions.

In addition, the width (the length in the direction of the major axis J32) of the internal space of the sheath 30 is designed to be substantially equal to the width of the implant main body 91, which helps ensure that frictional resistance between the implant 9 and the sheath 30 is lowered, an unnecessary force is not exerted on the implant 9, and the implant main body 91 can be disposed inside the sheath 30 in a sufficiently unfolded state. Note that the width of the internal space of the sheath 30 may be shorter than the width of the implant main body 91. In this case, the width of the sheath 30 is made smaller, which contributes to realization of a lower-invasiveness puncture member 3.

Note that in the following, for convenience of explanation, an end portion located on an inner side in the direction of the major axis J32 of the sheath 30 (on the proximal side with respect to the center) as shown in FIG. 6B will be referred to also as “inner circumferential portion A1,” an end portion located on an outer side in the direction of the major axis J32 (on the distal side with respect to the center) will be referred to also as “outer circumferential portion A2,” a surface oriented to the upper side in the figure will be referred to also as “front surface A3,” and a surface oriented to the lower side will be referred to also as “back surface A4.”

As shown in FIG. 6B, when a plane containing both a center point of the arc of the sheath 30 and a center point of the cross-sectional shape with respect to the longitudinal direction of the sheath 30 (a plane containing a center axis of the sheath 30) is made to be a plane f9, and the angle formed between the plane f9 and the minor axis J31 at a central portion S4 is made to be an inclination angle θ1, then the inclination angle θ1 is preferably an acute angle. With the inclination angle θ1 set to be an acute angle, the implant main body 91 can be disposed substantially in parallel to the urethra, as will be described later. Therefore, the urethra can be supported more effectively.

The inclination angle θ1 is not particularly limited, so long as it is an acute angle. For example, the inclination angle θ1 is preferably approximately 20 degrees to 60 degrees, more preferably 30 degrees to 45 degrees, and further preferably approximately 35 degrees to 40 degrees. By this setting, the above-mentioned effects can be further enhanced. Note that it is preferable that the above-mentioned numerical value range of the inclination angle θ1, for example, is satisfied at least at the central portion S4, and it is more preferable that the numerical value range is satisfied over substantially the whole region in the extending direction of the sheath 30. Here, the “central portion S4” refers to a region which at least includes a part located between a urethra and a vagina in a condition where the sheath 30 in an extended state as depicted in FIG. 6A is disposed inside a living body.

The configuration of the sheath 30 as above can also be expressed as follows. It can be said that, as illustrated in FIG. 6B, the sheath 30 is formed such that the major axis J32 is inclined against a center axis J5 of the arc, and the sheath 30 is configured such that the center axis J5 and an extension line J32′ of the major axis J32 have an intersection P. In this case, the angle θ5 formed between the center axis J5 and the extension line J32′ is equal to the inclination angle θ1. For example, the configuration of the sheath 30 can also be expressed as follows. As depicted in FIG. 6B, the sheath 30 can be said to include the inner circumferential portion A1, which is located at the inner circumferential edge thereof and has a minimum radius of curvature r1, and the outer circumferential portion A2, which is located at the outer circumferential edge thereof and has a maximum radius of curvature r2, in plan view as viewed from the direction of the center axis J5 (the axis J1), wherein the inner circumferential portion A1 and the outer circumferential portion A2 are located with a deviation from each other in the direction of the center axis J5.

In addition, the sheath 30 preferably is optically transparent such that the inside of the sheath 30 is visible from outside, which helps ensures that, for example, the state of a member inserted into the inside of the sheath 30 can be relatively easily confirmed from outside.

While the constituent material of the sheath 30 (the outer tube 301 and the inner tube 302) is not specifically restricted, the constituent material is preferably a rigid material such as to be able to maintain its shape and internal space in the state wherein the sheath 30 is inserted in a living body. Examples of such a rigid material applicable here include various resin materials such as polyethylene, polyimides, polyamides, polyester elastomers, polypropylene, etc. and various metallic materials such as stainless steel, aluminum or aluminum alloys, titanium or titanium alloys, etc. Note that adoption of a material other than a rigid material for constituting the sheath 30 may be made while, for example, reinforcing a wall of the sheath 30 with a reinforcement member. For instance, a braiding with a high strength may be embedded in the wall of the sheath 30, whereby the sheath 30 can maintain its shape and internal space in the state of being inserted in a living body. Another example of the reinforcement member may be a spiral body embedded in the wall of the sheath 30, whereby flexibility can be ensured while maintaining an internal space to such an extent that the implant 9 therein can be slid.

### Needle Body

As illustrated in FIG. 5, the needle body 39 can include a needle portion 391 which is tapered off, a proximal portion 392 provided on a proximal side of the needle portion 391, and a contact surface 393 provided at a boundary between the needle portion 391 and the proximal portion 392. With the proximal portion 392 fitted into a distal portion of the inner tube 302 of the sheath 30, the needle body 39 is interlocked to a distal portion of the sheath 30 in a freely detachable manner. With the proximal portion 392 thus fitted into the inner tube 302, the needle body 39 and the sheath 30 can be interlocked to each other with an appropriate force.

In a state in which the needle body 39 is interlocked to the sheath 30, the distal portion of the sheath 30 is in contact with the contact surface 393. By thus providing the needle body 39 with the contact surface 393 and fitting the needle body 39 into the inner tube 302 until the distal portion of the sheath 30 comes into contact with the contact surface 393, the needle body 39 can be appropriately interlocked to the inner tube 302. In addition, with the distal portion of the sheath 30 set or kept in contact with the contact surface 393, a positional deviation of the needle body 39 and the sheath 30 from each other can be prevented from being generated when the above-mentioned stress F1 is exerted thereon. Note that the contact surface 393 is preferably composed of a plane which is substantially orthogonal to the axis of the needle body 39.

In addition, the needle portion 391 has a flat cross-sectional shape having a major axis and a minor axis, like the sheath 30. In addition, as shown in FIG. 5, the needle portion 391 is disposed in such a manner that its major axis J42 and its minor axis J41 coincide substantially with the major axis J32 and the minor axis J31 of the sheath 30. By thus making the needle portion 391 have a flat shape, resistance at the time of puncturing a living body is reduced, and puncturing of a living body by the puncture member 3 can be performed relatively smoothly. Note that the flat shape of the needle portion 391 is not specifically restricted; like in the case of the sheath 30, examples of the applicable flat shape include an ellipse, a convex lens-like shape in section, a rounded-cornered rhombus, a rounded-cornered rectangle, and a fusiform with a central portion enlarged as compared with both end portions.

In addition, the needle portion 391 has an enlarged diameter portion (enlarged width portion) 391a which is the largest in outside diameter (width), namely, which is larger in outside diameter than portions on the distal side and the proximal side thereof, at an intermediate portion in the extending direction (axis direction) of the needle portion 391. Specifically, the needle portion 391 can include a gradually increasing outside diameter portion (gradually increasing width portion) 391b which is disposed on the proximal side and gradually increases in outside diameter (width) along the distal direction, a gradually decreasing outside diameter portion (gradually decreasing width portion) 391c which is disposed on the distal side of the gradually increasing outside diameter portion 391b and gradually decreases in outside diameter (width) along the distal direction, and the enlarged diameter portion 391a is located between the gradually decreasing outside diameter portion 391c and the gradually increasing outside diameter portion 391b. The enlarged diameter portion 391a functions also as a dissection portion for dissecting a living body tissue when the puncture member 3 punctures a living body, which helps ensure that puncturing of a living body by the puncture member 3 can be carried out relatively smoothly.

In addition, as shown in FIG. 5, the outside diameter of the enlarged diameter portion 391a is greater than the outside diameter of the distal portion of the sheath 30. Specifically, the width of the major axis J42 of the enlarged diameter portion 391a is greater than the width of the major axis J32 of the distal portion of the sheath 30, and the width of the minor axis J41 of the enlarged diameter portion 391a is greater than the width of the minor axis J31 of the distal portion of the sheath 30. This helps ensure that puncturing by the puncture member 3 can be carried out relatively smoothly. In accordance with an exemplary embodiment, in use of the puncture member 3, the needle body 39 punctures a living body, and the distal portion of the sheath 30 smaller than a punctured hole formed by the puncturing is gradually passed through the punctured hole, so that the distal portion of the sheath 30 is less liable to be caught on a wall portion of the punctured hole. Therefore, slidability of the puncture member 3 within the punctured hole can be enhanced, and puncturing of the living body by the puncture member 3 can be performed relatively smoothly. In accordance with an exemplary embodiment, since the distal portion of the sheath 30 makes contact with the contact surface 393 and the distal portion of the sheath 30 is hidden behind the contact surface 393 when viewed from the distal side of the needle body 39, the distal portion of the sheath 30 is further less liable to be caught on the wall portion of the punctured hole. Thus, in this embodiment, it is the needle body 39 that punctures a living body and dissects a living body tissue, and the sheath 30 substantially has neither a function of puncturing a living body nor a function of dissecting a living body tissue.

Note that, as will be explained in the description of a technique (procedure) later, the puncturing of a living body by the puncture member 3 is conducted with the inner tube 302 retracted in the outer tube 301 from the beginning to an intermediate point of the puncturing process, and is conducted with the inner tube 302 protruding from the outer tube 301 after the intermediate point of the puncturing process. Accordingly, “the outside diameter of the distal portion” refers to the outside diameter of the distal portion 301a of the outer tube 301 before the intermediate point of the puncturing process, and refers to the outside diameter of the distal portion of the inner tube 302 after the intermediate point of the puncturing process. To be more specific, therefore, for example, the outside diameter of the enlarged diameter portion 391a is greater than the outside diameter of the distal portion 301a of the outer tube 301, and the outside diameter of the distal portion 301a of the outer tube 301 is greater than the outside diameter of the distal portion of the inner tube 302.

Here, in this embodiment, for retracting the distal portion of the inner tube 302 into the outer tube 301, the outside diameter of the distal portion of the inner tube 302 is equal to or slightly smaller than the inside diameter of the outer tube 301. In the case of a configuration wherein, for example, the distal portion of the inner tube 302 is protruding from the outer tube 301 in an initial state, however, the distal portion of the inner tube 302 may be enlarged in diameter to be greater than the outside diameter of the distal portion 301a of the outer tube 301.

In addition, since the needle body 39 has the gradually increasing outside diameter portion 391b, the needle body 39 can exhibit the following effect. For example, in the case where a puncture route is deviated in the course of puncturing by the puncture member 3, it is necessary to retract the puncture member 3 within the punctured hole. In this case, if the gradually increasing outside diameter portion 391b is not formed, the proximal end of the needle portion 391 would behave like a “barb” and may be caught on a wall portion of the punctured hole, thereby making it impossible to retract the puncture member 3 smoothly. Where the needle portion 391 is provided with the gradually increasing outside diameter portion 391b, for example, the above-mentioned catching is restrained, so that the puncture member 3 can be retracted relatively smoothly.

The constituent material of the needle body 39 configured as above is not particularly limited; for example, the same or similar materials to those mentioned above for the sheath 30 can be used. In addition, the needle body 39 may be solid or may be hollow.

### Operation Member

The operation member 31 is a member for operating the sheath 30. As shown in FIG. 4, the operation member 31 can include a main body 32, and the pusher shaft (second shaft portion) 33. In addition, the main body 32 can include the stylet (first shaft portion) 321, a shaft portion 323, and an interlock portion 322 which interlocks the stylet 321 and the shaft portion 323. In such an operation member 31 as this, the stylet 321 and the pusher shaft 33 constitute a shaft portion 300, which can be extended.

In addition, the stylet 321 can be inserted into the sheath 30 together with the pusher shaft 33, and functions also as a reinforcement portion for reinforcing the sheath 30 from inside. Such a stylet 321 can have a substantially arcuate shape (curved shape) corresponding to the shape of the sheath 30. Note that a center angle and a radius of curvature of the stylet 321 are set in conformity with the center angle and the radius of curvature of the sheath 30. In addition, the shaft portion 323 extends on an axis J1 orthogonal to a plane f1 which intersects the center O of the stylet 321 and contains the stylet 321 (see FIG. 3). In addition, the interlock portion 322 interlocks a proximal portion of the stylet 321 and a distal portion of the shaft portion 323. The interlock portion 322 has a substantially L shape which is bent substantially at a right angle at an intermediate portion of the interlock portion 322.

In accordance with an exemplary embodiment, the main body 32 configured in this way is configured to be higher in rigidity than the sheath 30. The constituent material of the main body 32 is not specifically restricted, and examples of the applicable material include various metallic materials such as stainless steel, aluminum or aluminum alloys, titanium or titanium alloys, etc. Note that while the stylet 321, the shaft portion 323 and the interlock portion 322 are substantially the same in thickness (diametric size) in this embodiment, a configuration may be adopted wherein, for example, the interlock portion 322 and the shaft portion 323 are greater than the stylet 321 in thickness (diametric size) in order to sufficiently enhance rigidity of the main body 32 and to restrain the main body 32 from flexure or the like.

In addition, as shown in FIG. 3, a handle (operation unit) 324 is fixed to a proximal portion of the shaft portion 323. With the handle 324 rotationally operated, the operation member 31 can be rotationally operated about the axis J1. This enables smooth operation of the puncture apparatus 1. Note that the handle 324 may be omitted, and, in that case, the operation member 31 may be rotationally operated by grasping the interlock portion 322, for example. Note that in the following, rotation of the handle 324 at the time of rotationally moving the stylet 321 toward the distal side will be referred to also as “forward rotation,” and rotation of the handle 324 at the time of rotationally moving the stylet 321 toward the proximal side will be referred to also as “reverse rotation.”

In addition, as illustrated in FIGS. 4 and 5, a tip (interlock portion) 34 is provided at a distal portion of the stylet 321. In accordance with an exemplary embodiment, the tip 34 can have a first function to interlock the needle body 39 and the stylet 321 in a freely detachable manner, a second function to interlock the stylet 321 and the pusher shaft 33 in a slidable manner, and a third function to restrict the relative positional relation of the stylet 321 and the pusher shaft 33.

In the first place, the first function will be described. As shown in FIG. 5, the tip 34 has a main body portion 341 to which the stylet 321 is interlocked, and the main body portion 341 is provided at a distal portion thereof with a projection 35. In accordance with an exemplary embodiment, the proximal portion 392 of the needle body 39 is provided with an interlock hole 394 opening in an end face of the proximal portion 392 of the needle body 39. With the projection 35 inserted into the interlock hole 394 to interlock them, the needle body 39 and the stylet 321 can be interlocked through the tip 34 in a freely detachable manner. Thus, by interlocking the needle body 39 and the stylet 321 to each other, the stylet 321 and the sheath 30 are interlocked to each other through the needle body 39. Therefore, displacement of the sheath 30 relative to the stylet 321, specifically, for example, rotation and widthwise deviation and the like of the sheath 30 relative to the stylet 321 can be restrained. Accordingly, puncturing of a living body by the puncture member 3 can be performed smoothly.

The projection 35 is provided at a distal portion thereof with an elastically deformable portion 351 which can be contracted in diameter (contracted in width) as compared to a natural state through elastic deformation. The elastically deformable portion 351 can include two elastic pieces 352 and 353 spaced from each other, and a gap 354 provided between the elastic pieces 352 and 353. The elastically deformable portion 351 structured in this way contracts in diameter as compared to the natural state by elastic deformation of the elastic pieces 352 and 353 toward the inside (the gap 354). This configuration helps ensure a simplified configuration of the elastic deformation portion 351. Especially, for example, since distal ends of the elastic pieces 352 and 353 are free ends, in this embodiment, it is relatively easy for the elastic pieces 352 and 353 to be elastically deformed.

In addition, the elastic pieces 352 and 353 are provided with projecting portions 352a and 353a which project to the outside, and the outside diameter of the elastically deformable portion 351 is greater than the outside diameter of the portion on the proximal side thereof. The projecting portions 352a and 353a each have a roughly hemispheric shape such that their proximal-side surfaces are slant surfaces inclined toward the center axis of the projection 35.

In accordance with an exemplary embodiment, the interlock hole 394 can include an introduction portion 394a smaller than the outside diameter (width) of the elastically deformable portion 351 in the natural state, and an enlarged diameter portion (enlarged width portion) 394b which is located on the depth side with respect to the introduction portion 394a and is greater than the outside diameter (width) of the elastically deformable portion 351 in the natural state. In a condition where the projection 35 and the interlock hole 394 are interlocked, the elastically deformable portion 351 is located inside the enlarged diameter portion 394b, as depicted in FIG. 5. In this state, the elastically deformable portion 351 can be caught on a boundary portion between the enlarged diameter portion 394b and the introduction portion 394a, whereby the interlocked state of the projection 35 and the interlock hole 394 is maintained. When the tip 34 is pulled toward the proximal side with a force of not less than a predetermined value, in the interlocked state depicted in FIG. 5, the elastically deformable portion 351 is contracted in diameter, whereby the interlock (connection) is released, and the projection 35 is detached from the needle body 39.

Here, as aforementioned, in the case where the puncture route has been deviated in the course of puncturing by the puncture member 3 or in a similar case, it is necessary to retract the puncture member 3 and correct the puncture route to a proper puncture route. Since the retraction of the puncture member 3 is effected by reverse rotation of the handle 324, retraction of the stylet 321 is utilized to cause the needle body 39 and the sheath 30 interlocked to the stylet 321 to be retracted as one body with the stylet 321. In view of this, the strength of interlock (connection) between the projection 35 and the interlock hole 394 is designed in such a manner that the interlocked state of the stylet 321 and the needle body 39 is maintained even when the stylet 321 is retracted. Specifically, for example, the strength of interlock (connection) between the projection 35 and the interlock hole 394 can be relatively great as compared with the sliding friction of the living body tissue exerted on the surface of the sheath 30, and, therefore, the stylet 321 can be prevented from being detached from the needle body 39 when the puncture member 3 is drawn back. Accordingly, the stylet 321 can be prevented from being detached from the needle body 39 at the time of retraction.

Note that in the interlocked state shown in FIG. 5, the elastically deformable portion 351 is preferably in the natural state. Since the puncture apparatus 1 is in the interlocked state shown in FIG. 5 when in the natural state, the period of time for which the puncture apparatus 1 is in the interlocked state (for example, the period of time from the time of being manufactured to the time of being used) is long. Therefore, with the elastic pieces 352 and 353 being in the natural state when in the interlocked state, the period of time for which the elastic pieces 352 and 353 are elastically deformed can be greatly shortened, whereby a shape change (curl or kink) of the elastic pieces 352 and 353 and a lowering in elasticity of them can be prevented from occurring. As a result, the force necessary for canceling the interlocked state can be prevented from being lowered excessively, and unintended detachment of the needle body 39 from the stylet 321 can be effectively prevented from occurring.

Thus, the projection 35 and the interlock hole 394 have been described above. Note that while the tip 34 is provided with the projection 35 whereas the needle body 39 is provided with the interlock hole 394 in this embodiment, the disposition of the projection 35 and the interlock hole 394 is not limited to this. For example, the tip 34 may be provided with the interlock hole 394, and the needle body 39 may be provided with the projection 35. However, since the tip 34 is formed in a small thickness such that the tip 34 can be disposed inside the sheath 30, it may sometimes be difficult to secure a space for forming the interlock hole 394, depending on the size of the sheath 30 or the like factor. From the viewpoint of the degree of freedom in disposition, therefore, it can be preferable to provide the tip 34 with the projection 35 and to provide the needle body 39 with the interlock hole 394, as in this embodiment.

In the next place, the second function will be described. As depicted in FIG. 5, the main body portion 341 of the tip 34 is provided with a retaining portion 36 which retains the pusher shaft 33 in a slidable manner and guides the pusher shaft 33.

First, the pusher shaft 33 will be described. As illustrated in FIGS. 4 and 5, the pusher shaft 33 is disposed on the outer circumferential side of the stylet 321 in the state of being spaced from and aligned with the stylet 321, and is curved in a roughly arcuate shape along the arc of the stylet 321. In accordance with an exemplary embodiment, the pusher shaft 33 has a larger radius of curvature than that of the stylet 321, and is disposed concentrically with the stylet 321. Note that the center angle of the pusher shaft 33 is not particularly limited, and may be comparable to that of the stylet 321. Note that the pusher shaft 33 may be disposed on the inner circumferential side of the stylet 321.

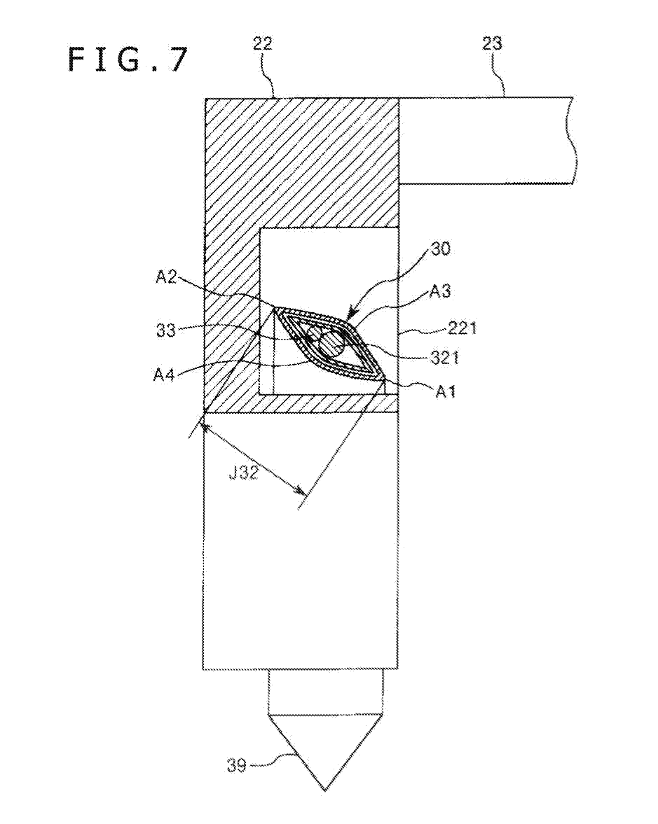

In addition, as shown in FIG. 7, the stylet 321 and the pusher shaft 33 are aligned with each other along the major axis J32 (the width direction) of the sheath 30. This helps enable efficient use of the internal space of the sheath 30, and it is therefore possible, for example, to make the stylet 321 and the pusher shaft 33 as large as possible in diametric size and to further enhance the rigidity of the operation member 31. In addition, the sheath 30 can be effectively reinforced from inside by the stylet 321 and the pusher shaft 33, and it is therefore possible to restrain the sheath 30 from unintended deformation. Note that in a condition where the sheath 30 is mounted in position, the stylet 321 is disposed substantially in the center of the sheath 30, and the pusher shaft 33 is disposed on the outer circumferential side of the sheath 30. Here, since the sheath 30 is thinner at its outer circumferential portion than at its central portion, a configuration wherein the diameter of the pusher shaft 33 is smaller than the diameter of the stylet 321 is preferably adopted accordingly. This helps enable effective prevention of unintended deformation of the sheath 30 in the condition where the sheath 30 is mounted in position.

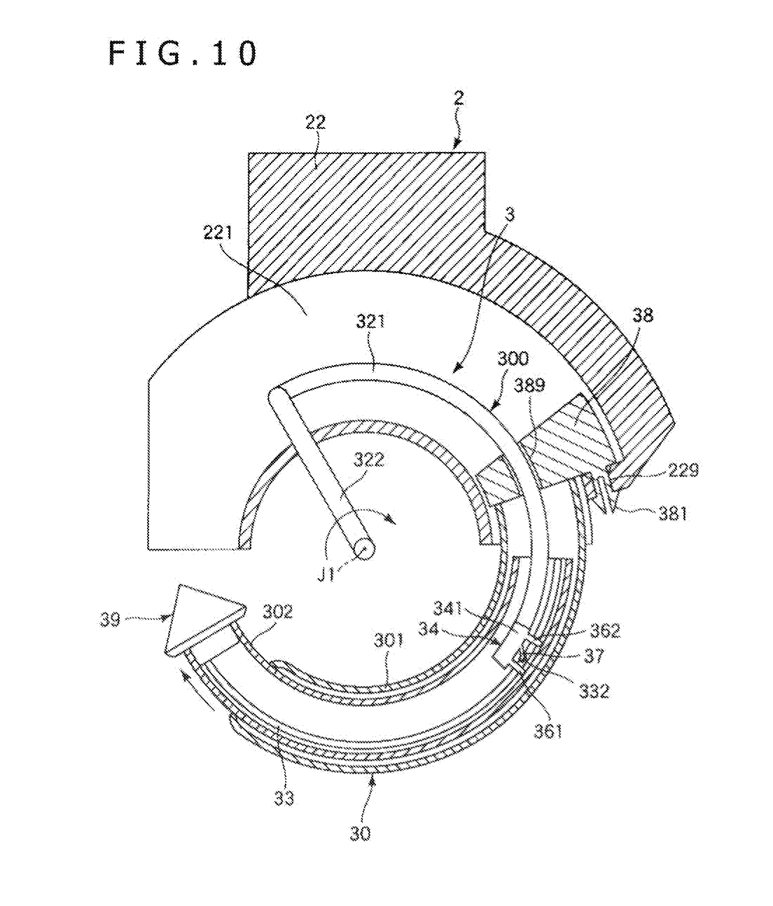

In accordance with an exemplary embodiment, as depicted in FIG. 5, a distal portion of the pusher shaft 33 can be fixed to the needle body 39. As will be described later, a step of pushing out the needle body 39 by the pusher shaft 33 to extend the sheath 30 (see FIGS. 10 and 21) is involved in the course of a procedure. In this connection, with the pusher shaft 33 fixed to the needle body 39, a pushing force of the pusher shaft 33 can be efficiently transmitted to the needle body 39, so that the needle body 39 can be pushed out efficiently. Accordingly, the procedure can be carried out relatively smoothly.

The pusher shaft 33 configured as above can be made to be higher in rigidity than the sheath 30. The constituent material of the pusher shaft 33 is not specifically restricted; for example, the same or similar materials to those mentioned above for the main body 32 can be used.

The retaining portion 36 for retaining the pusher shaft 33 configured as above can include a pair of guide portions 361 and 362 projecting to the outer circumferential side of the stylet 321, as shown in FIG. 5. These guide portions 361 and 362 are spaced from each other in the extending direction of the stylet 321. In addition, the guide portions 361 and 362 are formed in their distal portions with insertion holes 361a and 362a, and the pusher shaft 33 is inserted in and passed through these insertion holes 361a and 362a in a slidable manner. By this structure, the pusher shaft 33 is retained by the tip 34, and is slidable relative to the stylet 321.

Especially, for example, with the pusher shaft 33 retained by the two guide portions 361 and 362 spaced from each other along the sliding direction of the pusher shaft 33, as in this embodiment, rotation about the axis of the pusher shaft 33 as indicated by an arrow mark Z in FIG. 5 can be prevented. Since the pusher shaft 33 is curved in an arcuate shape as aforementioned, a rotation of the pusher shaft 33 relative to the stylet 321, if occurred, would make it impossible to slide the pusher shaft 33 in an intended direction. Note that in order to more effectively prevent the rotation of the pusher shaft 33, a configuration may be adopted wherein, for example, the cross-sectional shape of the pusher shaft 33 is a non-circular shape such as an ellipse, a tetragon, a triangle or the like, and the insertion holes 361a and 362a are shaped in conformity with the cross-sectional shape of the pusher shaft 33. In addition, in this embodiment, the above-mentioned rotation of the pusher shaft 33 can also be restrained by the sheath 30.

In addition, with the pusher shaft 33 retained by the two guide portions 361 and 362, the separated distance D1 between the tip 34 and the pusher shaft 33 can be maintained to be substantially constant (within a predetermined distance) between the guide portions 361 and 362 (between the insertion holes 361a and 362a). Therefore, the function (the third function) of a contact portion 37 described later can be exhibited. Note that from the viewpoint of keeping the separated distance D1 constant between the guide portions 361 and 362, the separated distance between the guide portions 361 and 362 (which varies depending on the rigidity of the pusher shaft 33 or the like factors) is preferably, for example, approximately 0.5 cm to 2.0 cm.

In the next place, the third function will be described. As shown in FIG. 5, the tip 34 is provided with the elastically deformable contact portion 37 for restricting a relative positional relation of the tip 34 with the pusher shaft 33. The contact portion 37 is obliquely projecting between the guide portions 361 and 362 toward the distal side of the pusher shaft 33 (the needle body 39 side).

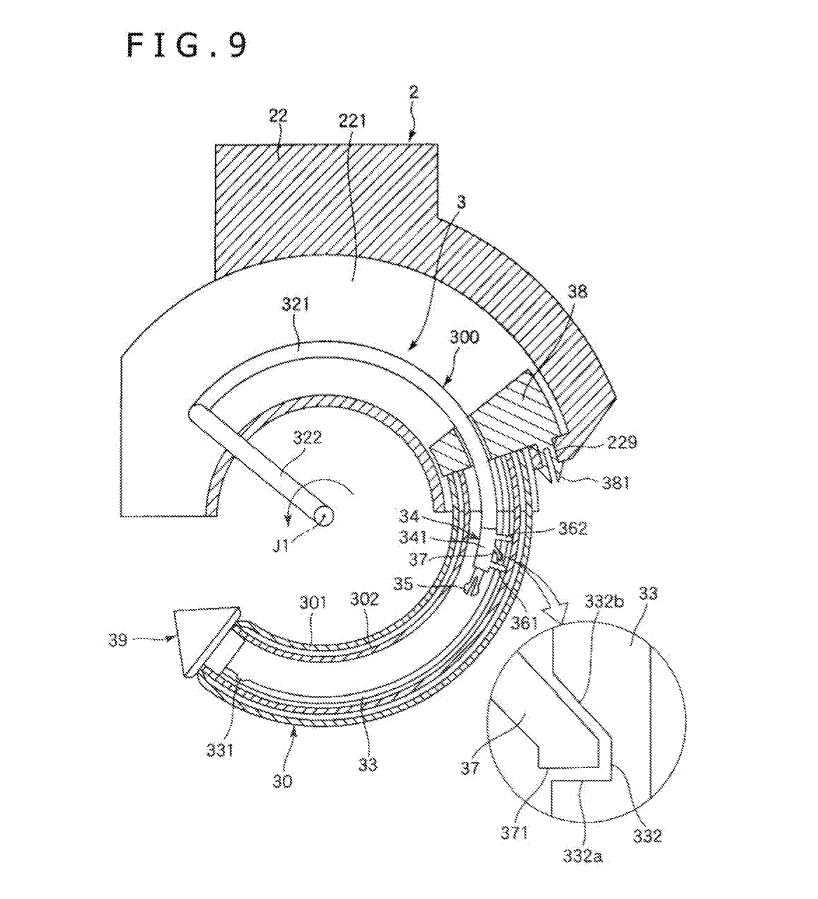

In accordance with an exemplary embodiment, the pusher shaft 33 is provided in its distal portion with a recess 331 as a relief portion, and is provided in its proximal portion with a recess 332 (see FIG. 9) as an engaging portion. In addition, in the initial state depicted in FIG. 5, a distal portion of the contact portion 37 is located inside the recess 331. In this state, the contact portion 37 is not elastically deformed but is in a natural state. In accordance with an exemplary embodiment, by preventing elastic deformation of the contact portion 37 in the initial state, the period of time for which the contact portion 37 is elastically deformed can be much shortened, whereby a shape change (curl or kink) of the contact portion 37 and a lowering in elasticity of the contact portion 37 can be prevented from occurring. As a result, the function of the contact portion 37 can be exhibited.

In addition, as will be described later, a step of sliding the stylet 321 proximally relative to the pusher shaft 33 to extend the shaft portion 300 is included in the course of a technique (procedure) conducted using the puncture apparatus 1 (see FIG. 9). In a state wherein the shaft portion 300 is thus extended, the distal portion of the contact portion 37 is located inside the recess 332. A surface on the distal side of the recess 332 is a contact surface 332a which is substantially orthogonal to the center axis of the pusher shaft 33 and is oriented toward the proximal side of the pusher shaft 33. In the extended state, the contact surface 332a and a distal end surface 371 of the contact portion 37 are facing each other or in contact with each other. In the extended state, therefore, distal movement of the stylet 321 relative to the pusher shaft 33 can be restricted. Accordingly, when the stylet 321 is moved distally, the pusher shaft 33 is moved distally together with (while maintaining a relative positional relation with) the stylet 321.

In accordance with an exemplary embodiment, since the contact portion 37 is provided in a region where the separated distance D1 between the tip 34 and the pusher shaft 33 can be kept constant (the region between the guide portions 361 and 362), as aforementioned, the distal end surface 371 of the contact portion 37 and the contact surface 332a contact each other, so that the above-mentioned function can be exhibited. Consequently, an erroneous operation of the puncture apparatus 1 and the like trouble can be prevented from occurring.

In accordance with an exemplary embodiment, a surface on the proximal side of the recess 332 is a slant surface 332b. Starting from the extended state, therefore, the stylet 321 can further be slid proximally relative to the pusher shaft 33, whereby the pusher shaft 33 can be detached from the tip 34. With the pusher shaft 33 configured to be thus detachable from the tip 34, the puncture member 3 can be easily removed from a living body.

Note that while the tip 34 is provided with the contact portion 37 whereas the pusher shaft 33 is provided with the recess 332 in this embodiment, the disposition of the contact portion 37 and the recess 332 is not limited to this. For example, the tip 34 may be provided with the recess 332, and the pusher shaft 33 may be provided with the contact portion 37. It is to be noted, however, that where the pusher shaft 33 is provided with the contact portion 37, such a design that the contact portion 37 does not contact the guide portion 362 when sliding the pusher shaft 33 must be adopted, so that the design may be complicated. From the viewpoint of a simple design, therefore, it can be preferable to provide the tip 34 with the contact portion 37 and to provide the pusher shaft 33 with the recess 332, as in this embodiment.

In the tip 34 as described above, it can be preferable that a portion having a flat shape is included at least at part in the extending direction of the main body portion 341 and that the portion having the flat shape functions to prevent rotation of the sheath 30. This helps make it possible to prevent the sheath 30 from being displaced or deformed in an unintended manner, and to puncture a living body by the puncture member 3 smoothly.

### Slide Lock Unit

The slide lock unit 38 is used for extending the shaft portion 300. As illustrated in FIG. 4, the slide lock unit 38 is slidably retained by the stylet 321. Specifically, for example, the slide lock unit 38 is provided therein with an insertion hole 389, and the stylet 321 is inserted in and passed through the insertion hole 389. In addition, by contact with the interlock portion 322, the slide lock unit 38 can be prevented from being detached from the proximal side of the stylet 321. In this configuration, when the handle 324 is rotated forwards from an initial state, the slide lock unit 38 is pushed by the interlock portion 322, and the slide lock unit 38 is moved distally together with the stylet 321, the pusher shaft 33 and the sheath 30.

In addition, the slide lock unit 38 is located on the proximal side of the sheath 30 and the pusher shaft 33, and is in contact with the proximal end of the sheath 30 at least. By this configuration, the sheath 30 can be prevented from slipping off proximally at the time of puncturing, and the sheath 30 can be prevented from being detached from the needle body 39. Note that the slide lock unit 38 may not be in contact with the proximal end of the sheath 30 but may be separated from the proximal end. In that case, however, it is preferable that the separated distance is shorter than the length (the depth of insertion into the inner tube 302) of the proximal portion 392 of the needle body 39. By this configuration, detachment of the sheath 30 from the needle body 39 can be prevented.

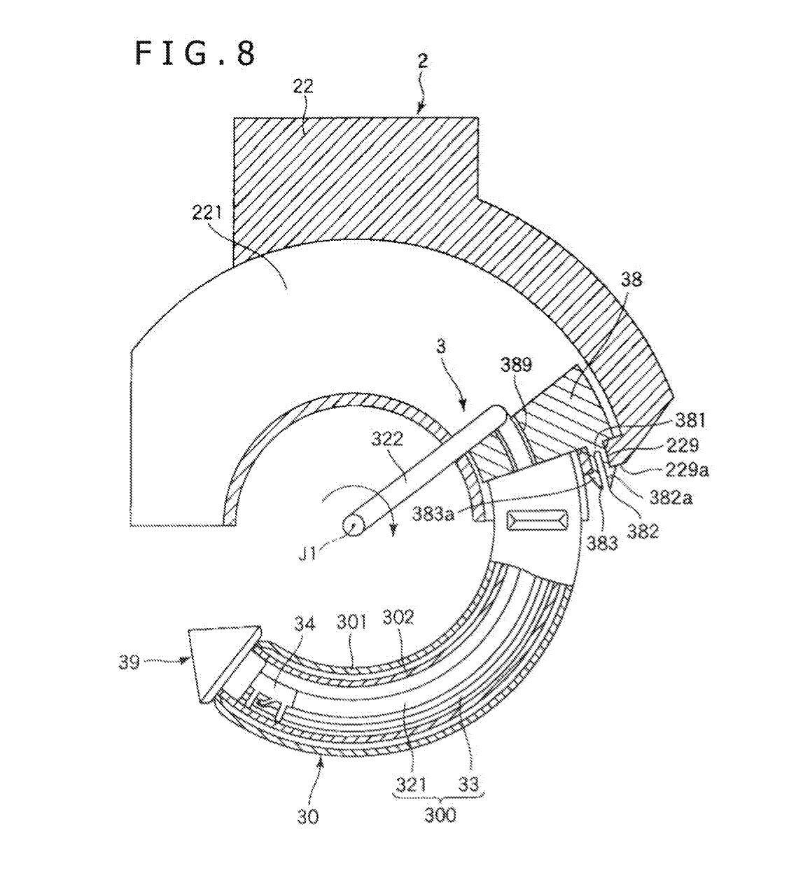

When the handle 324 is rotated forwards and the slide lock unit 38 is moved distally together with the shaft portion 300 and the sheath 30, starting from the initial state depicted in FIG. 4, the slide lock unit 38 is fixed to the frame 2, as shown in FIG. 8. Note that the fixation of the slide lock unit 38 to the frame 2 is effected by engagement between a projection (male-type engaging portion) 381 provided on the slide lock unit 38 and an engaging hole (female-type engaging portion) 229 provided in a guide portion 22 of the frame 2. A distal portion of the projection 381 can be divided into two elastic pieces 382 and 383, which are provided with claw portions 382a and 383a. When the projection 381 is inserted into the engaging hole 229, the claw portions 382a and 383a are passed through the engaging hole 229 and are engaged with an end face 229a of the engaging hole 229, the slide lock unit 38 is thereby fixed to the frame 2. This configuration helps enable the slide lock unit 38 to be fixed to the frame 2 while using a simple configuration. Especially, for example, in this embodiment, an end portion of the engaging hole 229 on the side of insertion of the projection 381 is tapered to widen the opening, whereby the projection 381 can be inserted into the engaging hole 229 relatively smoothly.

In addition, as illustrated in FIG. 8, the end face 229a is a surface fronting on the outside of the frame 2, and the claw portions 382a and 383a are exposed to the outside of the frame 2 in a state wherein the slide lock unit 38 is fixed to the frame 2. Therefore, the operator can visually confirm a state wherein the slide lock unit 38 is fixed to the frame 2. Accordingly, an erroneous operation of the puncture apparatus 1 can be effectively prevented. Furthermore, since the claw portions 382a and 383a are exposed to the outside of the frame 2, the operator can cancel the fixed state by pinching the claw portions 382a and 383a and pushing them into the inside. Therefore, operability of the puncture apparatus 1 can be enhanced. Note that the disposition of the projection 381 and the engaging hole 229 is not restricted to the disposition in this embodiment. A configuration may be adopted wherein the slide lock unit 38 is provided therein with the engaging hole 229 whereas the frame 2 is provided with the projection 381, on the contrary to this embodiment.

In a restriction state (the state of FIG. 8) in which the slide lock unit 38 is fixed to the frame 2, the interlock portion 322 is in contact with the slide lock unit 38, whereby the stylet 321 is restricted in moving further distally. In accordance with an exemplary embodiment, when the handle 324 is rotated reversely, the stylet 321 can be moved proximally while sliding relative to the slide lock unit 38. However, proximal movement of the sheath 30 can be restricted, since the proximal portion of the sheath 30 contacts the slide lock unit 38. In addition, both the needle body 39 interlocked to the sheath 30 and the pusher shaft 33 interlocked to the sheath 30 through the needle body 39 can also be restricted in proximal movement. Therefore, when the handle 324 is rotated reversely after the restriction state is established, only the stylet 321 is moved proximally, and the stylet 321 is detached from the needle body 39, as depicted in FIG. 9.

With only the stylet 321 thus moved proximally starting from the restriction state, the pusher shaft 33 is projected from the distal side of the stylet 321, resulting in an extended state wherein the shaft portion 300 is extended. In addition, as aforementioned, in the extended state, the contact portion 37 of the tip 34 is located inside the recess 332 of the pusher shaft 33, as shown in FIG. 9. In this state, distal sliding of the stylet 321 relative to the pusher shaft 33 is restricted. After the extended state is established, therefore, the shaft portion 300 can be moved distally, while maintaining the extended state, namely, while maintaining the relative positional relation between the stylet 321 and the pusher shaft 33. Therefore, when the handle 324 is again rotated forwards, the shaft portion 300 is moved distally while keeping the extended state and, attendantly, the needle body 39 is moved distally, as depicted in FIG. 10. In addition, in this instance, the inner tube 302 interlocked to the needle body 39 is also moved distally together with the needle body 39, whereby the inner tube 302 is protruded from the outer tube 301, to extend the sheath 30.

With such a slide lock unit 38 provided, the shaft portion 300 can be easily extended, and, further, the sheath 30 can be easily extended by the subsequent operation. Therefore, the technique (procedure) conducted using the puncture apparatus 1 can be performed relatively smoothly and accurately. For example, in this embodiment, when the stylet 321 is moved distally starting from the initial state, the slide lock unit 38 is automatically fixed to the frame 2, which permits a relatively easy operation of the puncture apparatus 1. In accordance with an exemplary embodiment, the slide lock unit 38 can be fixed to the frame 2 after the stylet 321 is moved distally starting from the initial state, as in this embodiment; therefore, a space for moving the stylet 321 is sufficiently secured on the proximal side of the stylet 321. Accordingly, proximal movement of the stylet 321 at the time of transition from the restriction state to the extended state can be performed relatively smoothly and assuredly.

### Frame

The frame 2 helps retain the puncture member 3 in a rotationally movable manner, and helps fix the insertion device 6 in a freely detachable manner. Such a frame 2 has a function of determining a puncture route for the needle body 39 when the puncture member 3 punctures a living body tissue. Specifically, for example, the frame 2 determines positional relations of the puncture member 3, the urethral-insertion device 4 and the vaginal-insertion device 5 such that when the puncture member 3 punctures a living body tissue, the needle body 39 passes between the urethral-insertion device 4 and the vaginal-insertion device 5 without colliding on any of them.

As shown in FIGS. 2 and 3, the frame 2 can include a bearing portion 21 which bears the shaft portion 323 of the puncture member 3, the guide portion 22 which guides the puncture member 3, an interlock portion 23 which interlocks the bearing portion 21 and the guide portion 22, and a fixation portion 24 which fixes the insertion device 6.

The bearing portion 21 is located on the proximal side of the puncture apparatus 1, and extends in a direction substantially orthogonal to the axis J1. The bearing portion 21 is formed therein with a through-hole 211 on the axis J1, and the shaft portion 323 is inserted in the through-hole 211 in a rotationally movable manner. By this, the puncture member 3 can be supported on the frame 2 in the state of being rotationally movable about the axis J1. Note that the handle 24 is disposed on the rear side of the bearing portion 21.

The guide portion 22 is located on the distal side of the puncture apparatus 1, and is disposed opposite to the bearing portion 21. As depicted in FIG. 4, the guide portion 22 has an arcuate guide groove 221 for guiding the puncture member 3, and the sheath 30 is disposed inside the guide groove 221. In addition, the aforementioned engaging hole 229 is provided at a distal portion of the guide groove 221. An opening 221a on the distal side of the guide groove 221 is an opening for protruding the needle body 39 to the outside, and an opening 221b on the proximal side is an opening for preventing the contact between the needle body 39 protruded to the outside and returned and the guide portion 22. The opening 221b on the proximal side opens widely in an outside surface and an upper surface of the guide portion 22, whereby the returned needle body 39 can be visually confirmed easily, and the contact between the returned needle body 39 and the guide portion 22 is effectively prevented. Further, the returned needle body 39 can be easily grasped.

In addition, in the initial state, the needle body 39 is in the state of protruding from the opening 221a on the distal side of the guide groove 221. By this, the position of the needle body 39 can be visually confirmed, so that positioning of the needle body 39 can be easily performed.

The interlock portion 23 interlocks the bearing portion 21 and the guide portion 22. In addition, the interlock portion 23 is in the form of a rod extending substantially in parallel to the axis J1. The interlock portion 23 can also function as a grasping unit. For example, the operator grasps the interlock portion 23 by one hand, and operates the handle 324 by the other hand, whereby the puncture apparatus 1 can be used in a stable state.

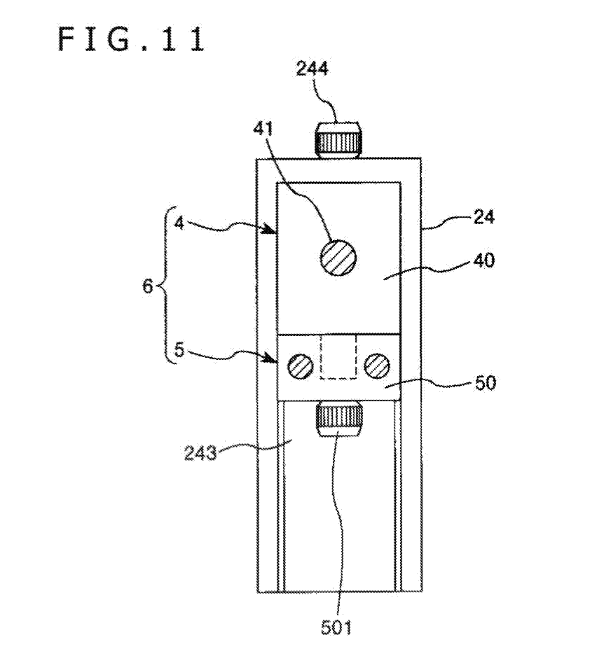

The fixation portion 24 is disposed opposite to the interlock portion 23, with the axis J1 therebetween. As depicted in FIG. 11, the fixation portion 24 can include a recess 243 into which to fit support portions 40 and 50 (described later) of the insertion device 6, and a male screw 244. In such a fixation portion 24, the insertion device 6 can be fixed to the fixation portion 24 by fitting the support portions 40 and 50 into the recess 243 and, further, fastening the male screw 244 into the support portion 40.

### Insertion Device

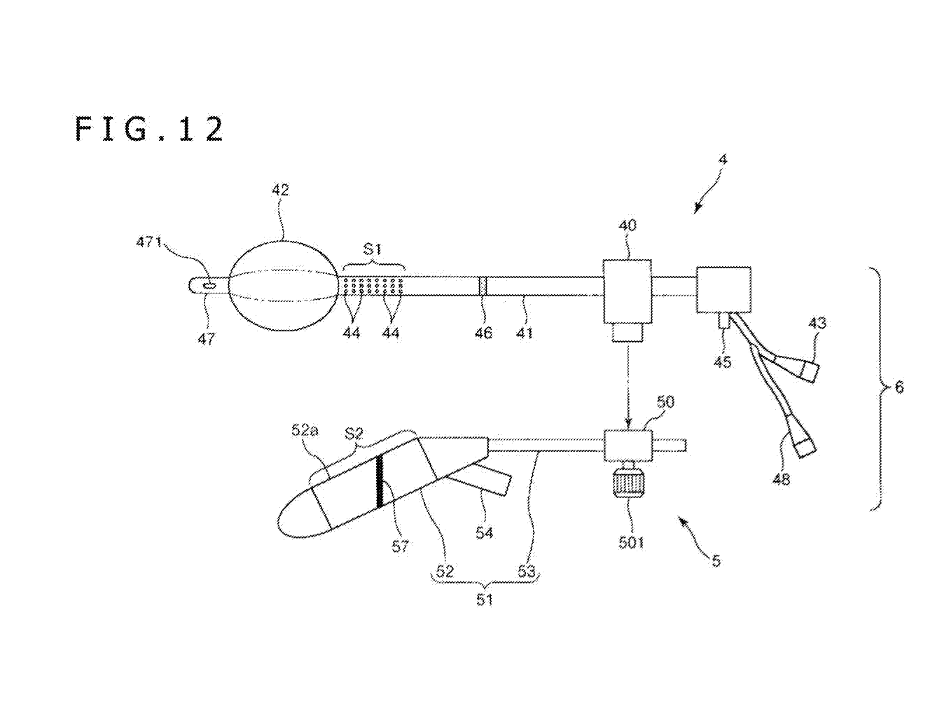

As illustrated in FIG. 12, the insertion device 6 can include the urethral-insertion device 4 and the vaginal-insertion device 5.

### Urethral-insertion Device

The urethral-insertion device 4 can include an elongate urethral-insertion portion 41 of which a part ranging from a distal end to an intermediate portion is to be inserted into a urethra, and the support portion 40 supporting the urethral-insertion portion 41. The constituent material or materials of the urethral-insertion portion 41 and the support portion 40 are not particularly limited. Examples of the usable materials include various metallic materials such as stainless steel, aluminum or aluminum alloys, titanium or titanium alloys, etc. and various resin materials.

The length of the urethral-insertion portion 41 (the portion on the distal side of the support portion 40) is not particularly limited, and is appropriately set according to the length of the patient's urethra or the shape of the patient's bladder. In view of the fact that the length of a general female urethra is, for example, approximately 30 mm to 50 mm, the length of the urethral-insertion portion 41 is preferably set to be, for example, approximately 50 mm to 100 mm, according to the fact.

In accordance with an exemplary embodiment, the urethral-insertion portion 41 is in the form of a straight tube. Such a urethral-insertion portion 41 is provided at its distal portion with a balloon 42 as an expansion body having extensibility and capable of expansion and contraction, and a urine drainage portion 47.

The balloon 42 is so disposed as to be located inside a bladder when the urethral-insertion portion 41 is inserted in a urethra. The balloon 42 is connected through the inside of the urethral-insertion portion 41 to a balloon port 43 provided at a proximal portion of the urethral-insertion portion 41. A balloon expanding device such as a syringe can be connected to the balloon port 43. When a working fluid (a liquid such as physiological salt solution, or a gas) is supplied from the balloon expanding device into the balloon 42, the balloon 42 is inflated. When the working fluid is drawn out of the balloon 42 by the balloon expanding device, In accordance with an exemplary embodiment, the balloon 42 is deflated. Note that in FIG. 12, the balloon 42 in a deflated state is indicated by alternate long and two short dashes line, while the balloon 42 in an inflated state is indicated by solid line.

The urine drainage portion 47 is used to drain urine present in the bladder in a state wherein the urethral-insertion portion 41 is inserted in the urethra. The urine drainage portion 47 is provided with a urine drain hole 471 providing communication between the inside and the outside of the urine drainage portion 47. The urine drain hole 471 is connected through the inside of the urethral-insertion portion 41 to a urine drain port 48 provided at a proximal portion of the urethral-insertion portion 41. Therefore, urine introduced via the urine drain hole 471 can be drained through the urine drain port 48.

The balloon 42 and the urine drainage portion 47 can be configured, for example, by a double lumen.

In addition, the urethral-insertion portion 41 is provided at its intermediate portion with a marker 46 for confirmation of the depth of insertion of the urethral-insertion portion 41 into a urethra. The marker 46 can be located, for example, at a urethral orifice when the urethral-insertion portion 41 is inserted in a urethra and the balloon 42 is located inside a bladder. This structure help enable the depth of insertion of the urethral-insertion portion 41 into the urethra to be easily confirmed. Note that the marker 46 is required only to be visually recognizable externally, and may be composed, for example, of a colored part, or a rugged (projected and recessed) part. Alternatively, in place of the marker 46, graduations accompanied by numerical values of distance from the distal end of the urethral-insertion portion 41 may be provided.

In addition, the urethral-insertion portion 41 is formed in its intermediate portion (on the distal side of the marker 46) with a plurality of suction holes 44. The suction holes 44 are arranged over the whole region in the circumferential direction of the urethral-insertion portion 41. Each of the suction holes 44 is connected through the inside of the urethral-insertion portion 41 to a suction port 45 provided in the support portion 40. A suction device such as a pump can be connected to the suction port 45. When the suction device is operated in a state wherein the urethral-insertion portion 41 is inserted in a urethra, a urethral wall can be attracted and fixed by suction onto the suction holes 44. Particularly, for example, with the plurality of suction holes 44 provided over the entire region in the circumferential direction of the urethral-insertion portion 41 as in this embodiment, a broad range of the urethral wall can be attracted and fixed by suction onto the urethral-insertion portion 41. Note that the number of the suction holes 44 is not particularly limited; for example, only one suction hole 44 may be provided. In addition, the layout of the suction holes 44 is not specifically restricted; for example, the suction holes 44 may be formed in only a part of the whole region in the circumferential direction of the urethral-insertion portion 41.

Note that when the urethral-insertion portion 41 is pushed in toward the inner side of a living body (toward the distal side of the urethral-insertion portion 41) in a state wherein the urethral wall is attracted and fixed by suction onto the urethral-insertion portion 41, the urethra and the bladder are accordingly pushed in toward the inner side of the living body, whereby the bladder can be shifted to such a position as not to overlap with a puncture route of the needle body 39. Therefore, the puncture route of the needle body 39 can be secured in a larger form, resulting in that puncturing by the puncture member 3 can be performed relatively accurately and safely.

In the next place, inclinations of the urethral-insertion portion 41 and the puncture member 3 will be described. As illustrated in FIG. 3, the axis J1 of the puncture member 3 is inclined against the axis J2 of the urethral-insertion portion 41 in such a manner that the separated distance between the axis J1 and the axis J2 increases along the distal direction. The inclination angle of the axis J1 in relation to the axis J2, for example, the inclination angle θ2 of the plane f9 (the plane f1) in relation to a plane f2 orthogonal to the axis J2, is not particularly limited, and is preferably, for example, approximately 20 degrees to 60 degrees, more preferably approximately 30 degrees to 45 degrees, and further preferably approximately 35 degrees to 40 degrees. As a result of this, puncturing by the puncture member 3 can be performed relatively easily, and the puncture distance by the puncture member 3 can be made shorter.

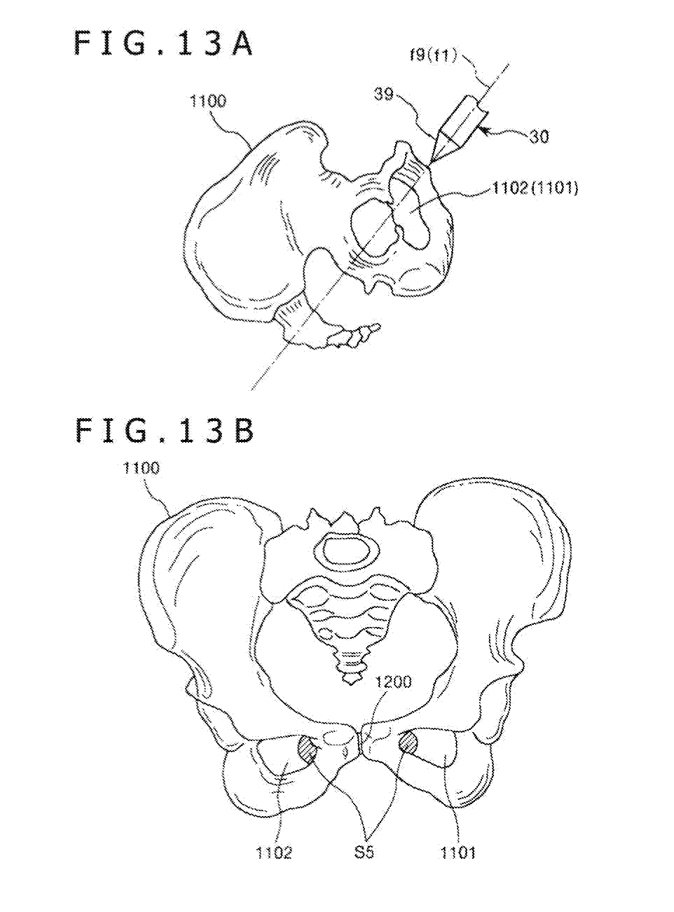

To be more specific, when the inclination angle θ2 is set within the above-mentioned range, as shown in FIG. 13A, it is possible for the needle body 39 to widely capture obturator foramens 1101 and 1102 of a pelvis 1100 on a planar basis and it is possible to widely secure a puncture space for the needle body 39. For example, with the patient set in a predetermined position (lithotomy position), the needle body 39 can be made to puncture in a direction comparatively nearer to the direction perpendicular to the obturator foramens 1101 and 1102. Consequently, puncturing by the needle body 39 can be carried out relatively easily.

When the needle body 39 thus punctures a living body tissue in a direction substantially perpendicular to the obturator foramens 1101 and 1102, the needle body 39 passes through a shallow portion of the tissue, so that the needle body 39 passes between the obturator foramens 1101 and 1102 by passing a shorter distance. Therefore, as shown in FIG. 13B, the needle body 39 can be made to pass through the obturator foramens 1101 and 1102 while passing near a pubic symphysis 1200, preferably through safety zones S5. The safety zone S5 is a zone where there are few nerves and blood vessels the damage of which is desired to be avoided. With the needle body 39 made to pass through the safety zones S5, therefore, puncturing by the needle body 39 can be performed safely. Consequently, the technique (procedure) conducted using the puncture apparatus 1 can be carried out with lowered invasiveness and with a reduced burden on the patient.

Thus, by setting the inclination angle θ2 to within the above-mentioned range, puncturing of a patient by the needle body 39 can be carried out suitably. In addition, by puncturing at the above-mentioned angle, a tissue between a mid-urethra (which refers to a middle portion in the lengthwise direction of the urethra) and the vagina can be easily aimed at as a target. Here, the position between the mid-urethra and the vagina is a position suitable as a part in which to embed the implant 9 for treatment of urinary incontinence. Taking the tissue between the mid-urethra and the vagina as a target, therefore, a more effective treatment can be performed.

Note that in the case where the inclination angle θ2 is below the above-mentioned lower limit or above the above-mentioned upper limit, a problem may arise depending on individual differences in the patient, the posture during a procedure, or the like factors. For example, it may be impossible for the needle body 39 to widely capture the obturator foramens 1101 and 1102 on a planar basis, or it may be impossible to sufficiently shorten the puncture route of the needle body 39.

The configuration of the urethral-insertion device 4 has thus been described above. In such a urethral-insertion device 4, the urethral-insertion portion 41 may not be slidable relative to the support portion 40 or may be slidable relative to the support portion 40. In the case where the urethral-insertion portion 41 is slidable relative to the support portion 40, a configuration may be adopted wherein, for example, when a screw (not illustrated) provided on the support portion 40 is untightened, the urethral-insertion portion 41 becomes slidable relative to the support portion 40, and when the screw is tightened, the urethral-insertion portion 41 becomes fixed to the support portion 40. According to such a configuration, the length of the urethral-insertion portion 41 can be adjusted, which helps enhance usability of the urethral-insertion device 4. This applies also to the vaginal-insertion device 5 described later.

While in the puncture apparatus 1 the urethral-insertion device 4 is fixed to the frame 2 such that the inclination angle θ2 is constant, this is not restrictive, and the inclination angle θ2 may be variable. Where the inclination angle θ2 is variable, the inclination angle θ2 can be controlled according to the individual differences in the patient and/or the posture during a procedure, which helps enhance usability of the puncture apparatus 1.

### Vaginal-Insertion Device

As illustrated in FIG. 12, the vaginal-insertion device 5 can include an elongate vaginal-insertion portion 51 of which a part ranging from a distal end to an intermediate portion is to be inserted in a vagina, and the support portion 50 supporting the vaginal-insertion portion 51. In addition, the vaginal-insertion portion 51 can include a distal portion 52 located on a distal side, and a shaft portion 53 interlocked to a proximal portion of the distal portion 52, the shaft portion 53 being supported by the support portion 50. In addition, the support portion 50 is provided with a male screw 501. With the male screw 501 fastened to a female screw (not illustrated) of the support portion 40, the vaginal-insertion device 5 can be fixed to the urethral-insertion device 4.

The constituent material or materials of the vaginal-insertion portion 51 and the support portion 50 are not particularly limited. Examples of the applicable materials include various metallic materials such as stainless steel, aluminum or aluminum alloys, titanium or titanium alloys, etc. and various resin materials, like in the case of the urethral-insertion device 4 (the urethral-insertion portion 41 and the support portion 40).

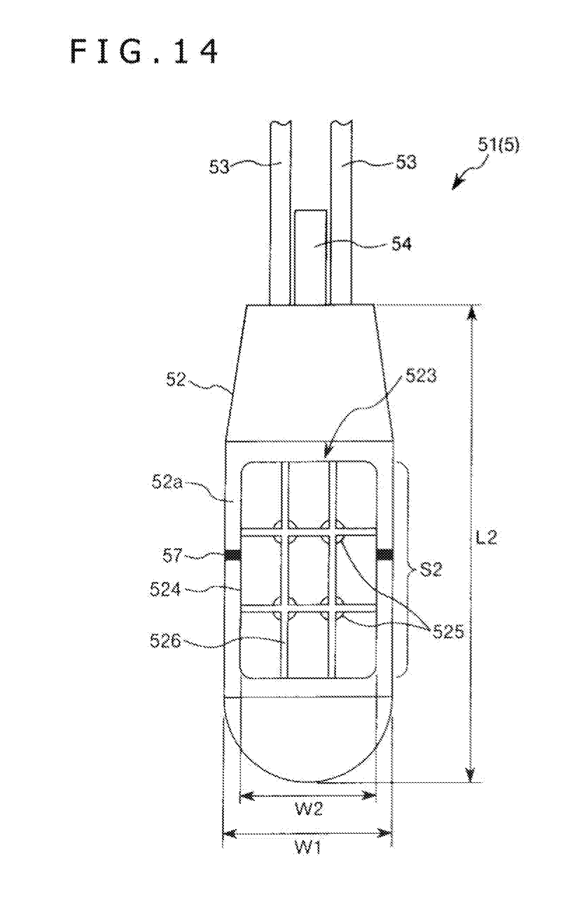

The distal portion 52 is a portion to be inserted into a vagina. As shown in FIG. 14, the distal portion 52 has a substantially constant width as a whole, and its distal part is rounded in shape. In addition, the distal portion 52 has a flat shape of being collapsed in the thickness direction. The length L2 of the distal portion 52 is not particularly limited, and is preferably, for example, approximately 20 mm to 100 mm, more preferably approximately 30 mm to 60 mm. The width W1 of the distal portion 52 is not specifically restricted, and is preferably, for example, approximately 10 mm to 50 mm, more preferably approximately 20 mm to 40 mm. When the length L2 and the width W1 of the distal portion 52 are set to within such ranges, the distal portion 52 is provided with a shape and size suitable for general vaginas. As a result, stability of the puncture apparatus 1 in the mounted state can be enhanced, and the burden on the patient can be lessened.

In addition, as illustrated in FIG. 12, the distal portion 52 has an upper surface (a surface on the urethral-insertion portion 41 side) 52a inclined against the urethral-insertion portion 41 in such a manner as to be spaced more from the urethral-insertion portion 41 as the distance from the distal end decreases. This structure helps ensure that the positional relation between the urethral-insertion portion 41 and the distal portion 52 can be made closer to the actual positional relation between a urethra and a vagina, as compared to the case where the upper surface 52a is not inclined in relation to the urethral-insertion portion 41. Therefore, in the mounted state, the puncture apparatus 1 is held onto the patient more stably, and the burden on the patient can be mitigated.

The inclination angle θ3 (see FIG. 3) of the upper surface 52a in relation to the urethral-insertion portion 41 is not particularly limited. For example, the inclination angle θ3 is preferably, for example, approximately 0 degrees to 45 degrees, and more particularly approximately 0 degrees to 30 degrees. This setting helps enable the above-mentioned effects to be exhibited more remarkably. In the case where the inclination angle θ3 is below the above-mentioned lower limit or above the above-mentioned upper limit, a problem may arise depending on individual differences in the patient, the posture during a procedure. For example, the vagina or the urethra may be deformed unnaturally in the mounted state, possibly resulting in that the puncture apparatus 1 cannot be held stably.

The upper surface 52a of the distal portion 52 as above is provided with a suction portion 523. The suction portion 523 can include a bottomed recess 524 opening in the upper surface 52a, a plurality of suction holes 525 provided at a bottom surface of the recess 524, and a grid-shaped rib 526 by which the inside of the recess 524 is partitioned into a plurality of regions. Each of the suction holes 525 is connected to a suction port 54 provided at a proximal part of the distal portion 52. In addition, the suction port 54 is provided in such a manner as to be located in the exterior of a living body in the mounted state.

The rib 526 is provided to rise from the bottom surface of the recess 524, and can include a plurality of wall portions extending in the lengthwise direction of the distal portion 52 and a plurality of wall portions extending in the widthwise direction of the distal portion 52. The plurality of suction holes 525 can be laid out in the manner of overlapping with intersections of the lengthwise-extending wall portions and the widthwise-extending wall portions. With the suction holes 525 laid out in this way, the suction holes 525 can be prevented from being closed with a vaginal wall getting into the recess 524 when the vaginal wall is attracted by suction. Therefore, attraction of the vaginal wall by suction can be performed assuredly. Note that the height of the rib 526 is not particularly limited. For example, the height of the rib 526 may be equal to the height of the recess 524, may be higher than the recess 524, or may be lower than the recess 524.

A suction device such as a pump can be connected to the suction port 54. When the suction device is operated in a state wherein the distal portion 52 is inserted in a vagina, a vaginal anterior wall is attracted and fixed by suction onto the distal portion 52. When the vaginal-insertion portion 51 is pushed in toward the inner side of the living body (toward the distal side of the vaginal-insertion portion 51) in a state wherein the vaginal anterior wall is attracted and fixed by suction onto the distal portion 52, the vagina can be pushed in accordingly. Therefore, the layout and/or shape of the vagina can be regulated, so that a puncture route for the needle body 39 can be secured sufficiently. As a result, puncturing by the puncture member 3 can be performed relatively accurately and safely.

Here, as shown in FIG. 12, a region S2 where the suction portion 523 is formed faces a region S1 where the suction holes 44 are formed. The puncture apparatus 1 is configured in such a manner that the needle body 39 passes between these regions S1 and S2. Since the urethral wall is attracted by suction onto the urethral-insertion portion 41 in the region S1 and the vaginal wall is attracted by suction onto the distal portion 52 in the region S2, it can be ensured that between the regions S1 and S2, the urethral wall and the vaginal wall are spaced apart from each other widely and assuredly. By passing the needle body 39 through such a zone, puncturing by the needle body 39 can be performed safely.

Note that the region S2 preferably extends over substantially the entire range in the width direction of the upper surface 52a, as depicted in FIG. 14. The width W2 of the region S2 is not particularly limited, and is preferably, for example, approximately 9 mm to 49 mm, more preferably approximately 19 mm to 39 mm. This helps ensure that the vaginal wall can be attracted by suction onto the distal portion 52 more assuredly, without being much affected by the shape of each patient's vaginal wall.

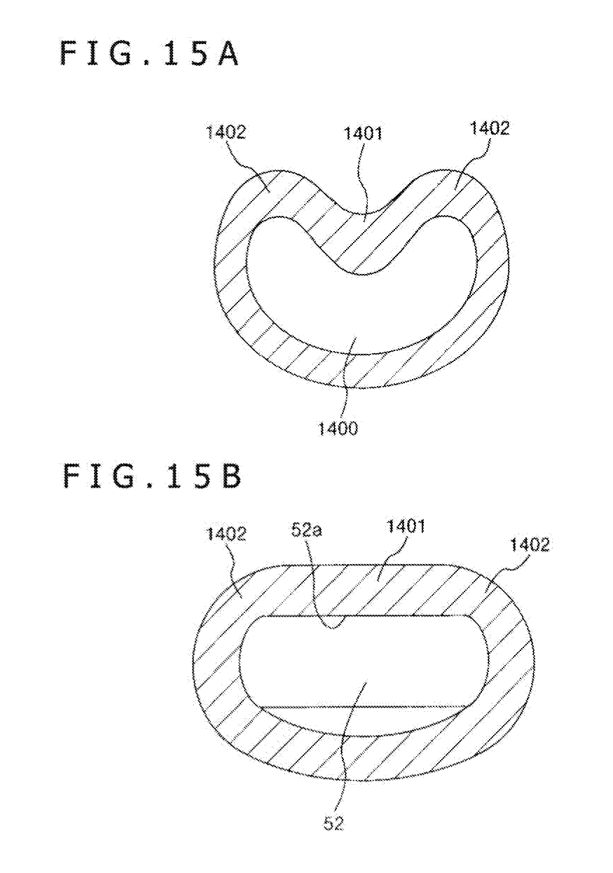

Particularly, for example, some patients may have a vagina 1400 in which a central portion 1401 of a vaginal anterior wall droops down into the vagina, as shown in FIG. 15A, to form hollowed portions (portions also called “button holes”) 1402 on both sides of the central portion 1401. Even in such a case, not only the central portion 1401 but also the hollowed portions 1402 can be attracted by suction with enhanced reliability, as depicted in FIG. 15B. With the hollowed portions 1402 thus attracted by suction onto the distal portion 52 more assuredly, the hollowed portions 1402 can be brought away from the urethra 1300, so that puncturing the hollowed portions 1402 by the needle body 39 by mistake can be effectively prevented.

In addition, the distal portion 52 can be provided with a marker 57 which help enable the puncture route of the needle body 39 to be confirmed. The marker 57 is provided such that the needle body 39 passes thereover. By confirming the position of the marker 57, therefore, the puncture route of the needle body 39 can be easily confirmed, and operability and safety of the puncture apparatus 1 can be enhanced. The marker 57 is preferably provided at least on a lower surface of the distal portion 52. The lower surface is oriented toward the vaginal orifice in the inserted state, and is a surface which can be visually checked by the operator by way of the vaginal orifice. With the marker 57 provided on the lower surface, therefore, the puncture route of the needle body 39 can be confirmed assuredly. The marker 57 is required only to be visually recognizable externally, and can be configured, for example, by a colored part or a rugged (projected and recessed) part.

The separated distance D2 (see FIG. 3) between the distal portion 52 configured in this manner and the urethral-insertion portion 41 is not particularly limited. The separated distance D2 is preferably set, for example, to be approximately 5 mm to 40 mm, in accordance with the separated distance between a urethral orifice and a vaginal orifice in general females.

The shaft portion 53 is in the form of a thin (diametrically small-sized) bar extending substantially in parallel to the urethral-insertion portion 41. The length of the shaft portion 53 (the separated distance between the distal portion 52 and the support portion 50) is not specifically restricted, and is preferably, for example, not more than approximately 100 mm, more preferably in the range of approximately 20 mm to 50 mm. By such a setting, the shaft portion 53 can be made to be appropriate in length, whereby operability of the puncture apparatus 1 is enhanced. If the length of the shaft portion 53 exceeds the above-mentioned upper limit, a problem may arise depending on the configuration of the frame 2 or the like factors. For example, the center of gravity of the puncture apparatus 1 may be spaced from the patient so largely that the stability of the puncture apparatus 1 in the mounted state is lowered.

3. Method of Using Puncture Apparatus

In the next place, a method of using the puncture apparatus 1, or a method of embedding the implant 9 into a living body by use of the puncture apparatus 1, will be described.

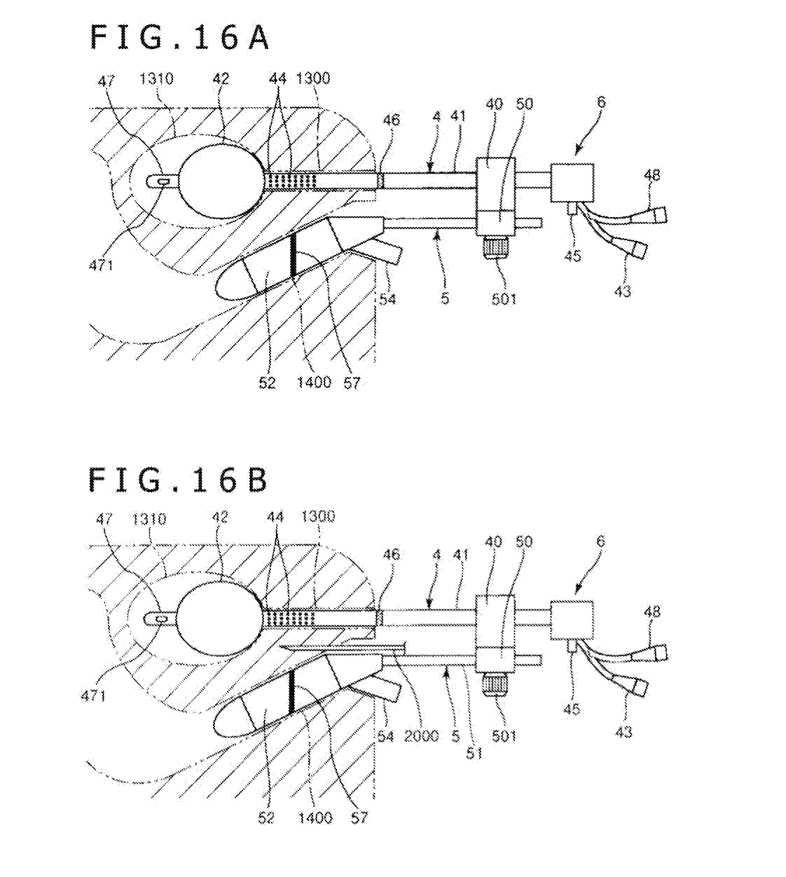

First, a patient is set into a lithotomy position on an operating table, and the insertion device 6 is mounted to the patient, as shown in FIG. 16A.

Specifically, first, the urethral-insertion portion 41 of the urethral-insertion device 4 is inserted into the urethra 1300, and the balloon 42 is disposed inside a bladder 1310. As a result, the urethra 1300 is rectified into a predetermined shape (rectilinear shape) by the urethral-insertion portion 41. Next, the balloon 42 is inflated, and urine is drained from the bladder 1310 through the urine drain hole 471 as required. In accordance with an exemplary embodiment, the distal portion 52 of the vaginal-insertion device 5 is inserted into the vagina 1400. Then, the puncture route of the needle body 39 is confirmed by use of the marker 57, after which the support portion 50 is fixed to the support portion 40. This completes mounting of the insertion device 6 onto the patient.

Next, suction devices are connected to the suction ports 45 and 54, and the suction devices are operated to attract and fix the urethral wall onto the suction holes 44 by suction, and to attract and fix the vaginal wall onto the suction portion 523 by suction. Here, for example when the urethral wall is properly attracted onto the suction holes 44 by suction, the suction holes 44 are closed with the urethral wall, so that suction through the suction port 45 is stopped or weakened. Similarly, when the vaginal wall is properly attracted onto the suction portion 523 by suction, suction through the suction port 54 is stopped or weakened. Therefore, on the basis of the conditions of suction through the suction ports 45 and 54 (for example, the magnitude of sounds generated by suction), the operator can check whether or not the urethral wall and the vaginal wall have been properly attracted onto the urethral-insertion portion 41 and the vaginal-insertion portion 51 by suction.

Note that the insertion device 6 may have a confirmation mechanism for mechanically confirming the attracted-by-suction state. The confirmation mechanism is not specifically restricted, so long as the mechanism helps enable the attracted-by-suction state to be confirmed. For instance, the confirmation mechanism may include a flow rate measurement unit (negative pressure gauge) for measuring flow rates through the suction ports 45 and 54, and a decision unit for deciding whether or not the attraction by suction has been properly performed, on the basis of the measurement results supplied from the flow rate measurement unit.

Subsequently, liquid dissection is conducted can be required. Specifically, for example, as illustrated in FIG. 16B, a puncture needle of a syringe 2000 is made to puncture the vaginal anterior wall from between the urethral-insertion portion 41 and the vaginal-insertion portion 51 (between the urethral orifice and the vaginal orifice), and a liquid such as physiological salt solution, a local anesthetic, is injected into a living body tissue between the urethra 1300 and the vagina 1400 (between the regions S1 and S2). This causes the living body tissue between the regions S1 and S2 to swell, whereby the urethral wall is pressed against the urethral-insertion portion 41, and the vaginal anterior wall is pressed against the distal portion 52.

Here, suction via the suction ports 45 and 54 is preferably continued also during the aforementioned liquid dissection. When the urethral wall is pressed against the suction holes 44 due to the liquid dissection, the urethral wall is brought into more secure contact with the suction holes 44, whereby the suction through the suction port 45 is stopped or weakened. Similarly, when the vaginal wall is pressed against the suction portion 523, the vaginal wall is brought into more secure contact with the suction portion 523, whereby the suction via the suction port 54 is stopped or weakened. On the basis of the conditions of suction through the suction ports 45 and 54, therefore, the operator can confirm whether or not the liquid dissection has been properly performed.

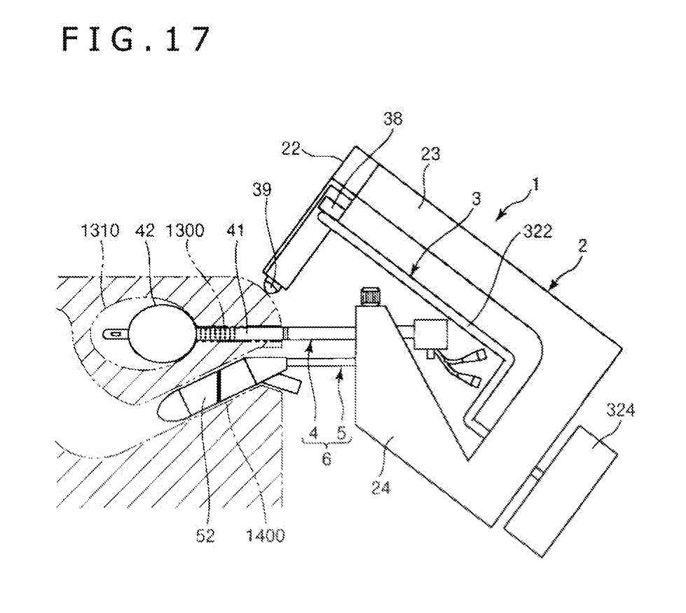

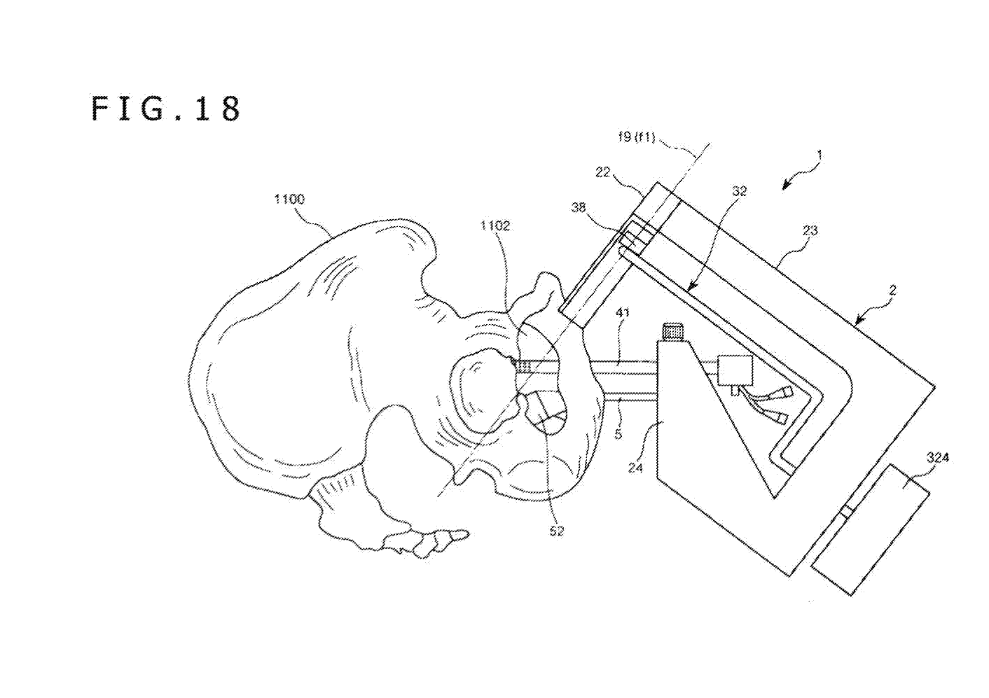

After the liquid dissection as aforementioned is performed to put the urethral wall and the vaginal wall into a sufficiently spaced apart state, the frame 2 can be fixed to the insertion device 6, as depicted in FIG. 17. This results in a mounted state in which the puncture apparatus 1 is mounted to the patient. Next, the puncture apparatus 1 is pushed in toward the inner side of the body. As has been described above, the urethral wall is attracted onto the urethral-insertion portion 41 by suction, and the vaginal wall is attracted onto the vaginal-insertion portion 51 by suction. When the puncture apparatus 1 is pushed in toward the inner side of the body, therefore, the urethra 1300 and the vagina 1400 are pushed in as well, so that the tissue therebetween is stretched. Therefore, sagging of the tissue can be reduced, and the shapes of the urethra 1300 and the vagina 1400 can be regulated, making it relatively easy for the needle body 39 to puncture the tissue. In addition, since the bladder 1310 can be pushed in toward the depth side as aforementioned, puncturing by the needle body 39 can be performed relatively safely. In this state, the positional relation between the pelvis 1100 and the puncture apparatus 1 is as illustrated in FIG. 18.

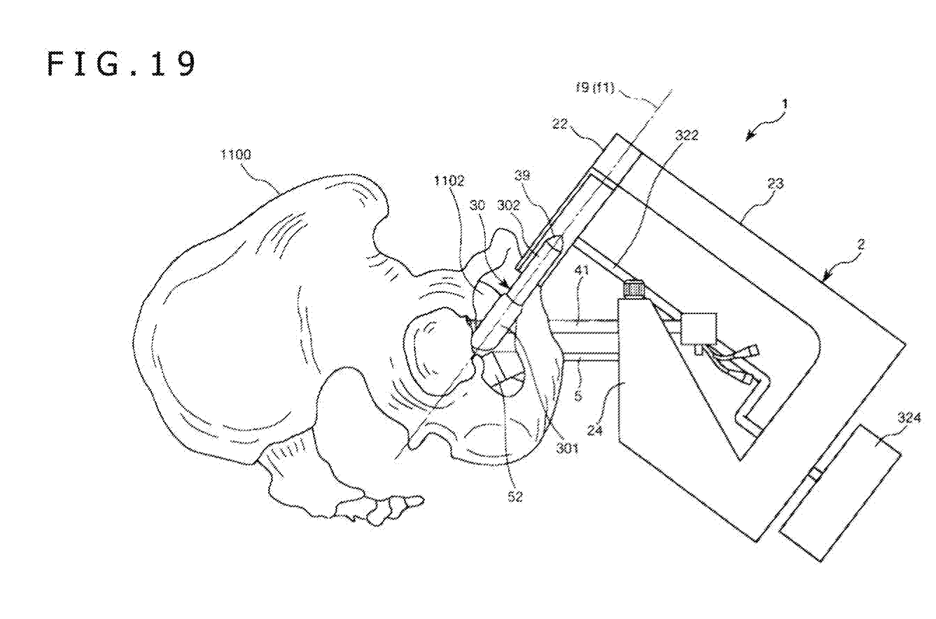

Next, in the state wherein the puncture apparatus 1 has been pushed in to the inner side of the body, the puncture apparatus 1 is positioned such that the puncture route of the puncture member 3 passes through the safety zones S5 in the obturator foramens 1101 and 1102 on the left and right sides of the pelvis 1100, and, while maintaining this condition, the handle 324 is rotated forwards, to result in the aforementioned restriction state, as depicted in FIG. 19. In this instance, for example, the needle body 39 enters the body by puncturing a body surface H of the patient's right-side inguinal region or neighboring part, and, for example, sequentially passes the obturator foramen 1101, a part between the urethra 1300 and the vagina 1400, and the obturator foramen 1102, to reach a position roughly beyond the obturator foramen 1102. In addition, the contact portion 301b provided on the outer tube 301 of the sheath 30 makes contact with the body surface H, resulting in a state wherein further insertion into the living body is restricted. Note that the position of the needle body 39 in this state is not particularly limited; for example, the needle body 39 may not yet have reached a position between the urethra 1300 and the vagina 1400.

With the needle body 39 thus moved forwards to a position of roughly beyond the obturator foramen 1102 by the puncturing in this step (first puncturing step), a longer distance inside the living body can be punctured in a condition where the rigidity of the puncture member 3 is relatively high (in a condition where the inner tube 302 is retracted in the outer tube 301 and where the sheath 30 is short). Therefore, the puncturing can be performed accurately and smoothly. Note that the position of the needle body 39 at the time when the first puncturing step is finished is not limited to the just-mentioned position; for example, the needle body 39 may have just moved to a position just in front of the obturator foramen 1102 after passing through a position between the urethra 1300 and the vagina 1400.

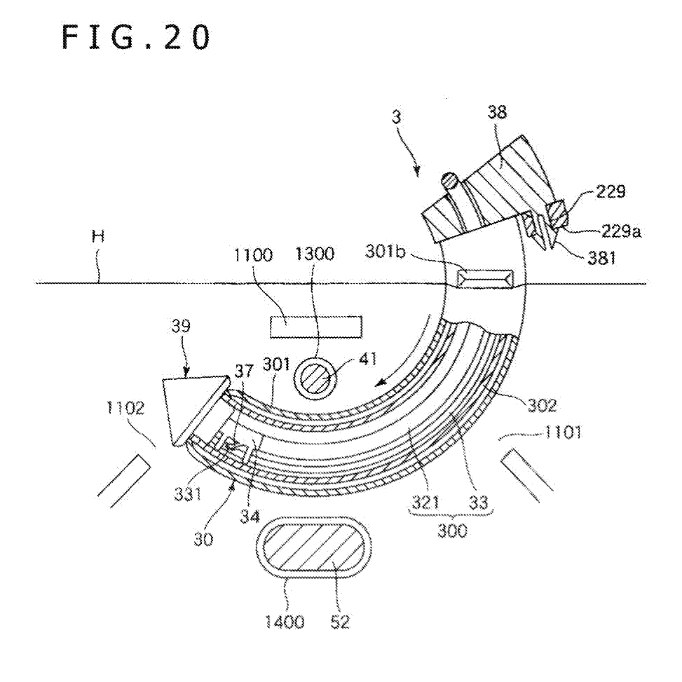

Subsequently, the handle 324 is rotated reversely, to put the shaft portion 300 into an extended state, as shown in FIG. 20. Next, the handle 324 is again rotated forwards, to move the shaft portion 300 toward the distal side, as depicted in FIG. 21. As a result, the needle body 39 is pushed by the pusher shaft 33, and the needle body 39 is moved toward the distal side together with the pusher shaft 33. In addition, the inner tube 302 connected to the needle body 39 is protruded from the outer tube 301 in the manner of being pulled by the needle body 39, whereby the sheath 30 is extended (second puncturing step).

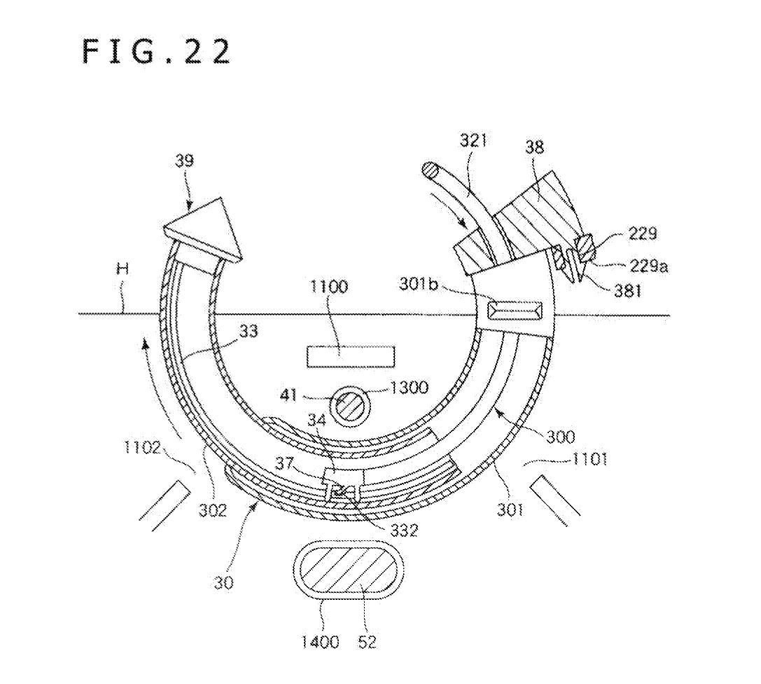

By this movement, the needle body 39 protrudes to the outside of the body via the body surface H of a left-side inguinal region or neighboring part thereof. In this case, by causing the puncture member 3 to puncture substantially perpendicularly in relation to the left and right obturator foramens 1101 and 1102 of the pelvis 1100, as aforementioned, a passage can be formed at a position suitable for the implant 9 to be placed indwelling. In this state, the positional relation between the pelvis 1100 and the puncture apparatus 1 is as illustrated in FIG. 22.

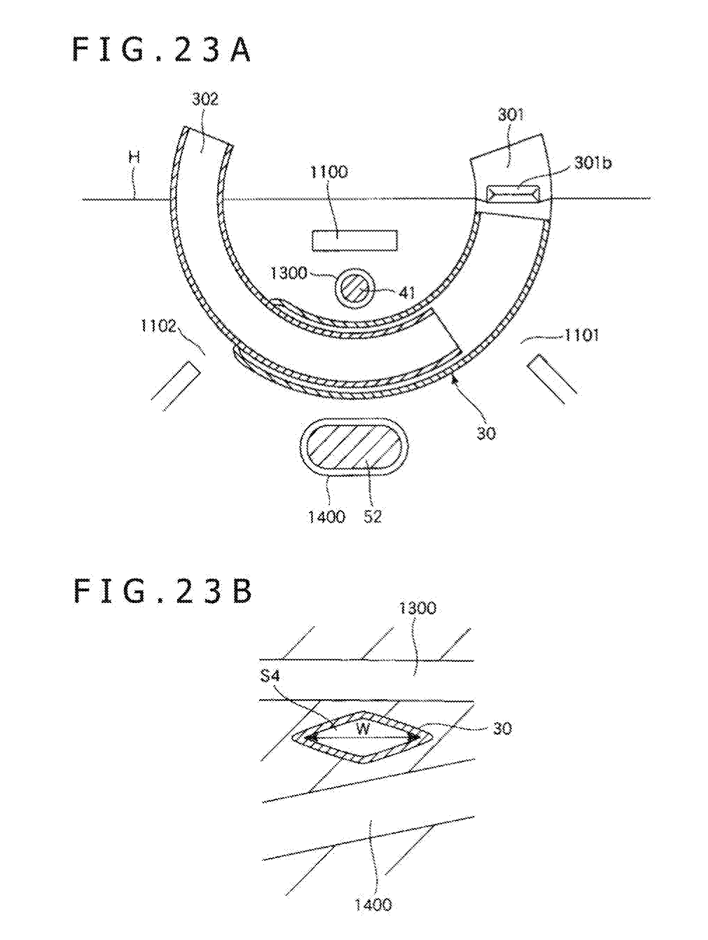

Next, with the sheath 30 kept disposed in the living body, the shaft portion 300 and the needle body 39 are drawn out of the sheath 30, and the frame 2 is detached from the insertion device 6. This results in a state wherein the sheath 30 is disposed in the living body, with the distal side opening and the proximal side opening exposed to the exterior of the living body, as illustrated in FIG. 23A. Note that this procedure is not particularly limited. For instance, a procedure may be adopted in which the handle 324 is first rotated reversely to draw out the stylet 321 through the proximal side opening of the sheath 30, then the frame 2 is detached from the insertion device 6, and finally the needle body 39 is removed from the inner tube 302 together with the pusher shaft 33. Alternatively, a procedure may be adopted in which the needle body 39 is first removed from the inner tube 302 together with the pusher shaft 33, then the handle 324 is rotated reversely to draw out the stylet 321 through the proximal side opening of the sheath 30, and finally the frame 2 is detached from the insertion device 6. Both of the procedures are realized owing to the configuration in which the stylet 321 and the pusher shaft 33 are freely detachable. Therefore, with the stylet 321 and the pusher shaft 33 configured to be freely detachable, the procedure can be performed smoothly.

Subsequently, the position of the sheath 30 is regulated as required. Specifically, the left and right lengths of protrusion of the sheath 30 are equalized, and the central portion S4 of the sheath 30 is thereby positioned between the urethra 1300 and the vagina 1400. In this state, as shown in FIG. 23B, the central portion S4 of the sheath 30 is disposed with its width direction (major axis J32 direction) W substantially in parallel to the urethra 1300. For example, the urethra 1300 rectified by the urethral-insertion portion 41 inserted therein and the width direction W of the central portion S4 of the sheath 30 are substantially parallel to each other.

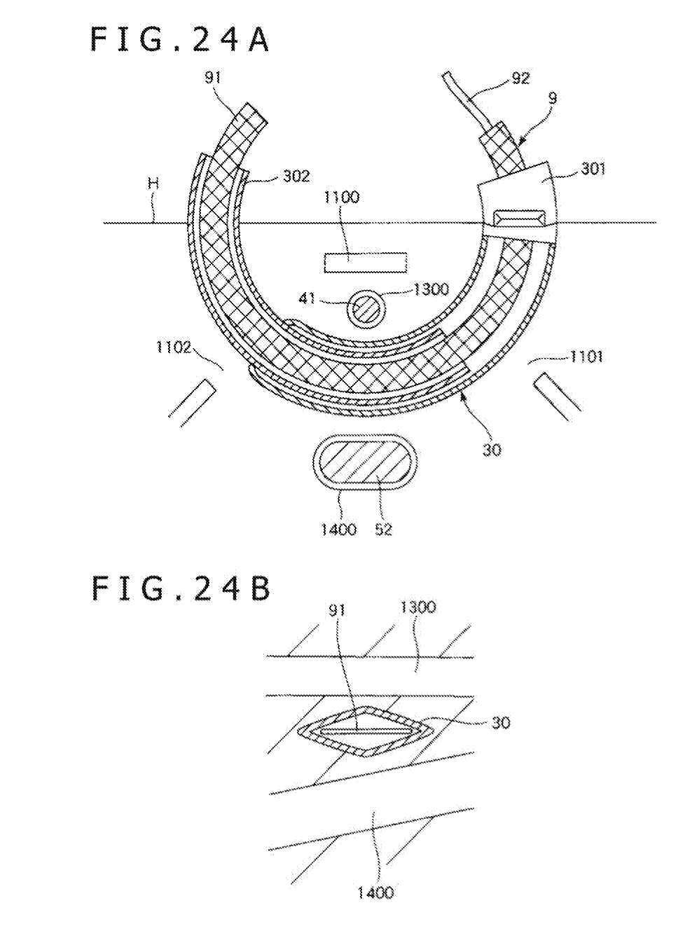

Next, while taking the implant 9 out of the wrapping material 90, the implant 9 is inserted into the sheath 30 to put the implant main body 91 into the state of protruding through the proximal side opening and the distal side opening of the sheath 30, as shown in FIG. 24A. The implant 9 is thus kept wrapped in the wrapping material 90 until immediately before disposed into the sheath 30, whereby the implant 9 can be prevented from contamination. Note that since the sheath 30 is flat in shape as has been described above, the posture of the implant main body 91 follows the flat shape of the sheath 30, so that the implant main body 91 is disposed in the sheath 30 with its width direction coinciding with the width direction of the sheath 30, as depicted in FIG. 24B. As for relation with the urethra 1300, the implant main body 91 is disposed in parallel to the urethra 1300 rectified in shape.

Subsequently, the attraction by suction of the urethral wall by the urethral-insertion portion 41 and the attraction by suction of the vaginal wall by the vaginal-insertion portion 51 are stopped. As a result, the positions and shapes of the urethra 1300 and the vagina 1400 are returned into their natural states. Next, the inner tube 302 is drawn distally out of the living body, and the outer tube 301 is drawn proximally out of the living body. In this case, the inner tube 302 and the outer tube 301 are moved in opposite directions and substantially simultaneously, and the inner tube 302 and the outer tube 301 are moved following arcuate paths along their shapes. By this, the sheath 30 is removed from the living body smoothly.

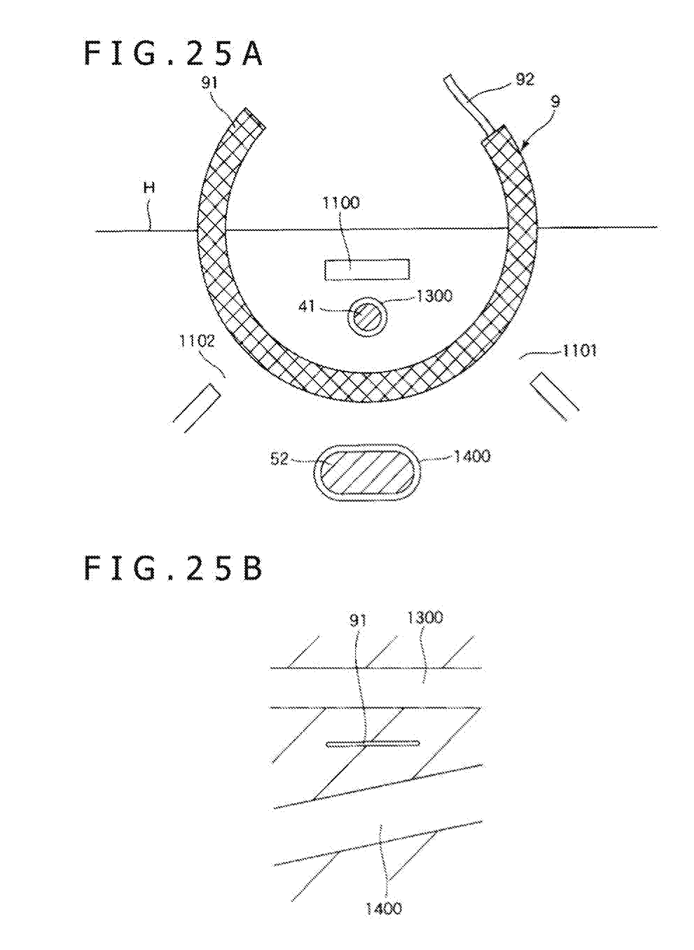

When the inner tube 302 and the outer tube 301 are gradually removed from the living body in the aforementioned manner, the surrounding tissues having been pushed open by the sheath 30 return to their original positions, and the tissues come into contact with the implant main body 91 gradually in the order from a central portion toward both end portions of the implant main body 91. As has been described above, the inner tube 302 and the outer tube 301 are moved in the directions along their shapes, and the sheath 30 has an internal space in which the implant main body 91 can be moved under sufficiently low friction. This enables the implant main body 91 to be placed indwelling as it is, without any unnecessary tension exerted thereon. Consequently, it is unnecessary to adjust the tension on the implant main body 91. As a result of the above operations, the implant main body 91 is left embedded in the living body, as shown in FIG. 25A. Note that in the state wherein the implant 9 is embedded in the living body, the implant main body 91 is disposed substantially in parallel to the urethra 1300, in the region between the urethra 1300 and the vagina 1400, as depicted in FIG. 25B. Therefore, the urethra 1300 can be supported by the implant main body 91 over a wider region.

By thus removing the sheath 30 from the living body by dividing the sheath 30, the sheath 30 can be drawn out of the living body easily. In addition, the outer tube 301 and the inner tube 302 being drawn out exert little influence on the posture of the implant main body 91 in the region between the urethra 1300 and the vagina 1400. Therefore, the implant main body 91 can be embedded in a desired posture. In addition, since the outer tube 301 and the inner tube 302 are drawn out of the living body with the urethral-insertion portion 41 inserted in the urethra 1300, it is possible to prevent an excessive tension from being exerted on the urethra 1300 by the implant main body 91 which is placed indwelling in the living body.

Next, the insertion device 6 is drawn out of the living body. Specifically, the urethral-insertion portion 41 is drawn out of the urethra 1300, and the vaginal-insertion portion 51 is drawn out of the vagina 1400. After the urethral-insertion device 4 is drawn out, the urethra 1300 returns into its shape in a natural state. Since the implant main body 91 is kept embedded in the living body tissue, however, the urethra 1300 in the natural state and the implant main body 91 can be maintained in the mutually parallel state.

Thereafter, unnecessary portions of the implant main body 91 are cut away, and the procedure is thereby finished.

As has been described above, according to the puncture apparatus 1, the sheath 30 can be extended, so that the center angle (length) of the sheath 30 can be made to be sufficiently large, and both end portions of the sheath 30 can be exposed from the body surface H assuredly, irrespectively of the patient's body type. Then, the implant main body 91 can be inserted easily and assuredly through both the end portions (both end openings) thus exposed.

In addition, in the initial state, the sheath 30 is in a contracted state, so that substantially the whole range of the sheath 30 can be accommodated in the guide groove 221. In accordance with an exemplary embodiment, in the case of using a sheath 30 which cannot be extended like in this embodiment and which has the same center angle (length) as that in the extended state from the beginning, the sheath 30 cannot be accommodated in the guide groove 221 completely. Therefore, a proximal portion of the sheath 30 protrudes from the guide groove 221, and such a problem as collision of the protruding proximal portion on the living body is generated, whereby operability of the puncture apparatus 1 is worsened.

In addition, placing the implant 9 indwelling can be coped with only a minimally invasive procedure such as puncturing by the puncture member 3, without need to perform a heavily invasive incision. Therefore, the burden on the patient is relatively light. In addition, safety for the patient is relatively high. Further, since the implant main body 91 can be embedded in parallel to the urethra 1300, the urethra 1300 can be supported over a wider region. In addition, the living body can be punctured by the needle body 39 while avoiding the urethra 1300 and the vagina 1400, and puncturing the urethra 1300 or the vagina 1400 by the needle body 39 by mistake can be prevented from occurring, so that safety can be secured. In addition, it is possible to avoid the problems arising from incision of the vagina as in the related art, such as the problem in which the implant 9 is exposed to the inside of the vagina from the wound caused by the incision, or the problem in which complications such as infection from the wound are generated. Thus, the procedure is highly safe, and the implant 9 can be reliably embedded.

While the puncture member according to the present disclosure has been described above with reference to the embodiments illustrated in the accompanying drawings, the present disclosure is not limited to the details of the above embodiments. The configurations of the components may be replaced by other arbitrary configurations having the same or equivalent functions. In addition, arbitrary structures may be added to the configurations according to the present disclosure.

In the above embodiments, description has been made of the case where the puncture apparatus is applied to an apparatus for use in embedding in a living body an implant embeddable for treatment of female urinary incontinence. The use of the puncture apparatus, however, is not limited to the described one.

For example, the target of the application of the present disclosure can include excretory disorders attendant on the weakening of the pelvic floor muscle group (urinary urgency, frequent urination, urinary incontinence, fecal incontinence, urinary retention, strangury or the like), and pelvic floor disorders including pelvic organ prolapse, vesicovaginal fistula, urethrovaginal fistula, pelvic pain or the like. In the pelvic organ prolapse, there are included disorders of cystocele, enterocele, rectocele, and hysterocele. Alternatively, there are included such disorders as anterior vaginal prolapse, posterior vaginal prolapse, vaginal apical prolapse, vaginal vault prolapse and the like in which the naming method thereof is based on the prolapsed vaginal-wall part.

Also, overactive tissues include bladder, vagina, uterus, and bowel. Less active tissues include bones, muscles, fascias, ligaments and the like. In particular, in the case of pelvic floor disorders, the less active tissues include an obturator fascia, a coccygeus fascia, a cardinal ligament, a uterosacral ligament, and a sacrospinous ligament.

For the procedure for interlocking an overactive tissue in the pelvic floor disorder with the less active tissue, there are included a retropubic sling surgery, a transobturator sling surgery (Transobturator Sling surgery, Transobturator Tape: TOT), a tension-free vaginal mesh (Tension-free Vaginal Mesh: TVM) surgery, a uterosacral ligament suspension (Uterosacral Ligament Suspension: USLS) surgery, a sacrospinous ligament fixation (Sacrospinous Ligament Fixation: SSLF) surgery, an iliococcygeus fascia fixation surgery, and a coccygeus fascia fixation surgery.

The puncture member disclosed herein can include: the needle body which punctures a living body; the shaft portion having the first shaft portion which is located on the proximal side of the needle body and is inserted into the living body together with the needle body; and the interlock portion which is provided at a distal portion of the first shaft portion and interlocks the first shaft portion and the needle body in a freely detachable manner. Therefore, the needle body and the first shaft portion are freely detachable from each other, so that the puncture member having punctured the living body can be easily removed from the living body. Accordingly, a procedure of embedding a living body tissue-supporting indwelling article into a living body can be performed relatively smoothly and safely.

Therefore, the puncture member disclosed herein does have industrial applicability.

Having described preferred embodiments of the present disclosure with reference to the accompanying drawings, it is to be understood that the disclosure is not limited to those precise embodiments and that various changes and modifications could be effected therein by one skilled in the art without departing from the spirit or scope of the disclosure as defined in the appended claims.

The detailed description above describes a puncture member. The invention is not limited, however, to the precise embodiments and variations described. Various changes, modifications and equivalents can effected by one skilled in the art without departing from the spirit and scope of the invention as defined in the accompanying claims. It is expressly intended that all such changes, modifications and equivalents which fall within the scope of the claims are embraced by the claims.

What is claimed is:

1. A puncture member comprising:
a needle body which punctures a living body;
a shaft portion which is located on a proximal side of the needle body and has a first shaft portion to be inserted into the living body together with the needle body; and
an interlock portion which is provided at a distal portion of the first shaft portion and interlocks the first shaft portion and the needle body in a freely detachable manner.

2. The puncture member according to claim 1, wherein when the first shaft portion is moved proximally in a condition where the needle body is located in the living body, the needle body is moved proximally together with the first shaft portion while retaining a state of being interlocked with the first shaft portion.

3. The puncture member according to claim 1, further comprising
a restriction unit which restricts proximal movement of the needle body while permitting proximal movement of the first shaft portion; and
wherein the needle body and the first shaft portion are detached from each other when the first shaft portion is moved proximally in a condition where proximal movement of the needle body is restricted by the restriction unit.

4. The puncture member according to claim 1,
wherein one of the needle body and the interlock portion is provided with a projection, whereas the other of the needle body and the interlock portion is provided with an interlock hole for interlocking with the projection; and
the first shaft portion and the needle body are interlocked to each other by insertion of the projection into the interlock hole.

5. The puncture member according to claim 4,
wherein the projection includes an elastically deformable portion which can be contracted in width as compared to a natural state by elastic deformation;
the interlock hole includes an introduction portion having a width smaller than the width of the elastically deformable portion in the natural state, and an enlarged width portion which is located on a depth side of the introduction portion and is greater in width than the introduction portion; and
the first shaft portion and the needle body are interlocked to each other, by the elastically deformable portion being located in the enlarged width portion.

6. The puncture member according to claim 5, wherein the interlock portion includes a flat-shaped portion.

7. The puncture member according to claim 1, wherein the shaft portion includes the first shaft portion and a second shaft portion slidable relative to the first shaft portion, and the shaft portion is extendable by sliding of the first shaft portion and the second shaft portion.

8. The puncture member according to claim 1, comprising
a pipe-shaped medical tube in which the shaft portion is inserted, and the needle body is connected to a distal portion of the medical tube.

9. The puncture member according to claim 8,
wherein the medical tube includes an outer tube and an inner tube, the inner tube disposed inside the outer tube and is slidable relative to the outer tube, the medical tube being extendable by sliding of the outer tube and the inner tube; and
wherein the needle body is connected to a distal portion of the inner tube.

10. A method of forming a path in living body tissue, the method comprising:
puncturing the living body tissue with the puncture member of claim 1.

11. A puncture apparatus comprising:
a needle body;
a shaft portion including a first shaft portion which is located on a proximal side of the needle body and is interlocked to the needle body in a freely detachable manner and a second shaft portion interlocked to the first shaft portion in a slidable manner;
a restriction unit establishing a restriction state in which proximal movement of the second shaft portion is restricted while proximal movement of the first shaft portion is permitted; and
a support portion which supports the shaft portion.

12. The puncture apparatus according to claim 11, wherein the restriction unit is movable relative to the support portion in an initial state, and comes into the restriction state by being fixed to the support portion.

13. The puncture apparatus according to claim 12, wherein the restriction unit is fixed to the support portion when the first shaft portion and the second shaft portion are both moved distally starting from the initial state.

14. The puncture apparatus according to claim 11, wherein the restriction unit is interlocked to the first shaft portion in a slidable manner.

15. The puncture apparatus according to claim 11, further comprising:
an interlock portion which is interlocked to a proximal portion of the first shaft portion and restricts detachment of the restriction unit from a proximal side of the first shaft portion, and the restriction unit is moved distally together with the first shaft portion by being pushed by the interlock portion.

16. The puncture apparatus according to claim 11, comprising:
a male-type engaging portion provided on one of the restriction unit and the support portion; and
a female-type engaging portion provided in the other of the restriction unit and the support portion, and the restriction unit is fixed to the support portion by engagement between the male-type engaging portion and the female-type engaging portion.

17. A puncture member comprising:
a needle body;
a shaft portion including a first shaft portion which is located on a proximal side of the needle body and is interlocked to the needle body in a freely detachable manner, and a second shaft portion provided in alignment with the first shaft portion; and
an interlock portion which is provided at a distal portion of the first shaft portion, retains the second shaft portion in a slidable manner, and restricts relative positional relation between the first shaft portion and the second shaft portion.

18. The puncture member according to claim 17, wherein the interlock portion maintains a separated distance between the first shaft portion and the second shaft portion within a predetermined distance in a vicinity of the interlock portion.

19. The puncture member according to claim 17, wherein the interlock portion has at least a pair of insertion holes spaced apart along an extending direction of the second shaft portion, the second shaft portion is inserted in and passed through the pair of insertion holes, and the separated distance between the first shaft portion and the second shaft portion is maintained within the predetermined distance between the pair of insertion holes.

20. The puncture member according to claim 17, wherein the interlock portion restricts distal movement of the first shaft portion relative to the second shaft portion after an extended state in which the shaft portion is extended is established by proximal movement of the first shaft portion relative to the second shaft portion starting from an initial state.
